(19) United States
(12) Patent Application Publication
(10) Pub. No.: US 2026/0027305 A1
(43) Pub. Date: Jan. 29, 2026

(54) Coated element and method for coating an element

(71) Applicant: SCHOTT PHARMA AG & CO. KGAA, Mainz (DE)

(72) Inventors: Tanja WOYWOD, Mainz (DE); Inga GHECZY, Mainz (DE); Jovana DJORDJEVIC-REISS, Mainz (DE); Thorsten SCHNEIDER, Mainz (DE)

(21) Appl. No.: 18/997,621

(22) PCT Filed: Jul. 26, 2023
(86) PCT No.: PCT/EP2023/070699

§ 371 Date: Jan. 22, 2025

(30) Foreign Application Priority Data
Jul. 29, 2022 (EP) 22187836.6

(51) Int. Cl. A61M 5/31; A61K 9/00; A61K 31/7088; C23C 16/40; C23C 16/511

(52) U.S. Cl. CPC A61M 5/3129; A61K 9/0019; A61K 31/7088; C23C 16/401; C23C 16/511; A61M 2205/0222; A61M 2205/0238; A61M 2207/00

(57) ABSTRACT

This disclosure relates to a coated element having a surface, wherein at least a part of the surface of the element is coated by a coating having a smoothing layer, a barrier layer, and a protection layer. A system or kit, including the coated element, a closed system comprising the coated element, and a method for coating an element.

This disclosure relates to a coated element having a surface, wherein at least a part of the surface of the element is coated by a coating, a system or kit, comprising the coated element, a closed system comprising the coated element, and a method for coating an element.

### BACKGROUND

Pharmaceutical compositions for injection, for example vaccines, are commonly administered by means of a syringe to a patient. Therefore, the pharmaceutical composition might be accommodated in a vial and is only drawn in the syringe a few minutes before injection, or alter-natively the pharmaceutical composition is directly filled in a syringe after production and is accommodated in the syringe until administration. In case the pharmaceutical composition is directly filled in the syringe after production, the pharmaceutical composition is in direct contact with the inner surface of the syringe over a long time. Thereby, the pharmaceutical composition may interact with the surface of the syringe or air might diffuse through the wall of the syringe. As a consequence, the efficacy of the pharmaceutical composition might decrease or is contaminated with extractables and leachables. To decrease the interaction and suppress the diffusion of air, the surface of the syringe might be coated with a coating.

### SUMMARY OF THE INVENTION

However, these coatings are commonly very sensitive to high or low pH values and may itself contaminate the pharmaceutical composition if the coating peels of or dissolves. The inventors recognized these issues and thus, it is an object of the present application to provide a coated element having sufficient barrier properties and improved resistance and a method for the production said coated element. In detail, it is an object of the present disclosure to provide a coated element exhibiting one or more of:
improved barrier properties with regard to air, especially oxygen;
improved pH resistance;
reduced amount of extractables;
reduced amount of leachables;

reduced leachable species;
reduced delamination;
improved adhesion of the coating or the coating and the décor; and/or
improved gliding properties of the coating.

In addition, it is an object of the present disclosure to provide an improved method for the production of a coated element. In detail, it is an object of the present disclosure to provide a method for the production of a coated substrate, preferably according to any embodiment described herein, exhibiting one or more of:
increased applying speed;
reduces costs;
increased production reliability; and/or
reduced production fluctuations.

In one aspect, this disclosure relates to a coated element having a surface, wherein at least a part of the surface of the element is coated by a coating, wherein the coating comprises, in this order:
a smoothing layer (SL);
a barrier layer (BL); and
a protection layer (PL),
   wherein the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1*10⁵, wherein [SiC₃H₉⁺]_{SL} are the counts of [SiC₃H₉⁺] ions in the smoothing layer, measured by a TOF-SIMS (Time-of-Flight Secondary-Ion-Mass-Spectrometry), wherein [Si₂O⁺]_{SL} are the counts of [Si₂O⁺] ions in the smoothing layer, measured by a TOF-SIMS, and/or
   wherein the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1*10⁵, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and/or
   wherein the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10¹⁰, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS,
   wherein the coated element optionally has a gliding force of 0.1 to 10.0 N, and/or a breaking force of 1 N to 18 N.

Cluster ion beams, in combination with a surface-sensitive analytical technique such as X-ray photoelectron spectroscopy (XPS) or secondary-ion mass spectrometry (SIMS), provide access to depth profiles of organic materials while retaining molecular information. In contrast to atomic ions, cluster ions do not penetrate deeply into target materials and, therefore, the energy of their impact is deposited within the first few nanometers of the surface, resulting in high sputtering yields and minimal subsurface damage. This allows the measurement of the distribution of organic materials through micrometers of industrially and medically relevant materials with depth resolutions in the region of 10 nm (Shard et al., Argon Cluster Ion Beams for Organic Depth Profiling: Results from a VAMAS Interlaboratory Study; Anal. Chem. 84, 2012, 7865-7873).

The coated elements according to this disclosure were characterised using a cluster ion beam assay in combination with secondary-ion mass spectrometry (SIMS) (see the ‘ToF-SIMS’ part in the ‘Methods’ section) which proves the improved layer structure obtained according to the methods according to this disclosure.

In another aspect, this disclosure relates to a coated element having a surface, wherein at least a part of the surface of the element is coated by a coating, wherein the coating comprises, in this order:
a smoothing layer (SL);
a barrier layer (BL); and
a protection layer (PL),
   wherein the smoothing layer has a thickness of 1 nm or more, and/or
   wherein the barrier layer has a thickness of 1 nm or more, and/or
   wherein the protection layer has a gliding force of 0.1 to 10.0 N, and/or a breaking force of 1 N to 18 N, and/or
   wherein the coated element has a gliding force of 0.1 to 10.0 N, and/or a breaking force of 1 N to 18 N.

It is advantageous if the coating comprises a smoothing layer (SL), a barrier layer (BL) and a protection layer (PL), because the interaction of a pharmaceutical composition and the surface of the element may then be significantly reduced. The smoothing layer (SL) provides for an optimal contact and interaction with the (coated) element. The barrier layer (BL) is characterised by the predominance of inorganic species which may be provided by high temperatures in combination with oxygen employed during the coating process, e.g. using a PICVD method which generates a plasma. The barrier layer (BL) strongly reduces the oxygen transmission rate within and across the coating. The protection layer (PL) may be chemically similar to the smoothing layer (SL) and protects the barrier layer (BL).

In another aspect, this disclosure relates to a coated element having a surface, wherein at least a part of the surface of the element is coated by a coating, wherein the coating comprises, in this order:
a smoothing layer (SL);
a barrier layer (BL); and
a protection layer (PL),
   wherein b1_{PL}≤[SiC₂H⁺]_{PL}/[SiO⁺]_{PL}≤b2_{PL}, wherein b1_{PL} is 0.5, wherein b2_{PL} is 1*10⁵, wherein [SiC₂H⁺]_{PL} are the counts of [SiC₂H⁺] ions in the protection layer, measured by a TOF-SIMS; and wherein [SiO⁺]_{PL} are the counts of [SiO⁺] ions in the protection layer, measured by a TOF-SIMS.

In another aspect, this disclosure relates to a coated element having a surface, wherein at least a part of the surface of the element is coated by a coating, wherein the coating comprises, in this order:
a smoothing layer (SL);
a barrier layer (BL); and
a protection layer (PL),
   wherein b1_{BL}≤[SiO⁺]_{BL}/[SiC₂H⁺]_{BL}≤b2_{BL}, wherein b1_{BL} is 1, wherein b2_{BL} is 1*10⁵, wherein [SiC₂H⁺]_{BL} are the counts of [SiC₂H⁺] ions in the barrier layer, measured by a TOF-SIMS, wherein [SiO⁺]_{BL} are the counts of [SiO⁺] ions in the barrier layer, measured by a TOF-SIMS.

In another aspect, this disclosure relates to a coated element having a surface, wherein at least a part of the surface of the element is coated by a coating, wherein the coating comprises, in this order:
a smoothing layer (SL);
a barrier layer (BL); and
a protection layer (PL),
   wherein b1_{SL}≤[SiC₂H⁺]_{SL}/[SiO⁺]_{SL}≤b2_{SL}, wherein b1_{SL} is 1.0, wherein b2_{SL} is 1*10⁵, wherein [SiC₂H⁺]_{SL} are the counts of [SiC₂H⁺] ions in the smoothing layer, measured by a TOF-SIMS, wherein [SiO⁺]_{SL} are the counts of [SiO⁺] ions in the smoothing layer, measured by a TOF-SIMS.

In another aspect, this disclosure relates to a coated element having a surface, wherein at least a part of the surface of the element is coated by a coating, wherein the coating comprises, in this order:
a smoothing layer (SL);
a barrier layer (BL); and
a protection layer (PL),
   wherein the thickness of the smoothing layer is 1 nm to 20000 nm, or 5 nm to 5000, or 100 nm to 2000 nm, or 150 nm to 1500 nm, or 200 nm to 1000 nm, or 250 nm to 500 nm.

In a further aspect, this disclosure relates to a system or kit, comprising the coated element according to this disclosure, wherein the element is a syringe or cartridge; a plunger, preferably a plunger rod; and optionally a closure, preferably a tip cap and/or a needle shield.

In a further aspect, this disclosure relates to a closed system comprising:
the coated element according to this disclosure, wherein the element is a syringe or cartridge;
a plunger, preferably a plunger rod; and
a closure, preferably a tip cap and/or a needle shield,
   wherein the closed coated element passes the container closure integrity test according to DIN EN ISO 8871-5:2016; chapter 4.4 in combination with Annex D.

In a further aspect, this disclosure relates to a method for coating an element, preferably a coated element according to this disclosure, comprising the steps:
(1) providing an element comprising a surface;
(2) applying a smoothing layer;
(3) applying a barrier layer;
(4) applying a protection layer; and
(5) optionally applying a gliding layer,
wherein each of the steps (2) to (4) comprises surrounding at least part of the surface of the element with a precursor, and
wherein each of the steps (2) to (4) comprises irradiating the precursor to generate a plasma using a PICVD method,
wherein the step (3) comprises establishing a flow rate of O₂ of 10 sccm or more during the irradiation of the precursor.

Advantageously, the method provides for coating an element, wherein the coated element comprises, preferably in this order, a smoothing layer, a barrier layer, a protection layer, and optionally a gliding layer. The method advantageously allows and provides for designing and adjusting the respective application steps such that the smoothing layer, the barrier layer, the protection layer, and optionally the gliding layer may be physically and/or chemically distinct. The method is versatile and may be adapted such that e.g. the barrier layer may predominantly display inorganic surface moieties and/or may have a very low oxygen transmission rate, and/or such that e.g. the protection layer protects the barrier layer, and/or such that e.g. the gliding layer (if present) may predominantly display organic surface moieties.

### DESCRIPTION OF THE FIGURES

FIG. 1A shows a schematic depiction of the layer structure of a coated element (CE) according to one embodiment of this disclosure, including a smoothing layer (SL), a barrier layer (BL), a protection layer (PL), and an optional gliding layer (GL), wherein the coated element may have a polymer substrate. The four different layers, i.e. SL, BL, PL and optionally GL, cover the coated element (CE) at least partially.

FIG. 1B shows a schematic depiction of the TOF-SIMS data for the positively charged ion species SiO⁺, SiC₂H⁺ and C₃H₅⁺ and indicates how the smoothing layer (SL), the barrier layer (BL), the protection layer (PL), and the gliding layer (GL) on the coated element (CE) are assigned based on this data. At the interface GL/PL, the point-to-point normalization values markedly change for the SiO⁺ and the SiC₂H⁺ species. At the interface PL/BL, the point-to-point normalization values markedly change for the SiO⁺ and the SiC₂H⁺ species. In the BL, the point-to-point normalization values become maximal for SiO⁺, whereas the point-to-point normalization values become minimal for SiC₂H⁺. In the coated element (CE), the species C₃H₅⁺ becomes dominant, i.e. the point-to-point normalization values become maximal.

FIG. 1C shows a schematic depiction of the TOF-SIMS data for the negatively charged ion species SiCH₃O⁻, SiO³⁻ and C₄H₃⁻ and indicates how the smoothing layer (SL), the barrier layer (BL), the protection layer (PL), and the gliding layer (GL) on the coated element (CE) are assigned based on this data. At the interface GL/PL, the point-to-point normalization values markedly change for the SiO³⁻ and the SiCH₃O⁻ species. At the interface PL/BL, the point-to-point normalization values markedly change for the SiO³⁻ species. In the BL, the point-to-point normalization values become maximal for SiO³⁻, whereas the point-to-point normalization values become minimal for SiCH₃O⁻. In the coated element (CE), the species C₄H₃⁻ becomes dominant, i.e. the point-to-point normalization values become maximal.

FIGS. 2A to 2G shows the results of example 1 of the herein described TOF-SIMS measurement (positive ions) displaying the species C₃H₅⁺ (FIG. 2A), CH₃⁺ (FIG. 2B), Si₂O⁺ (FIG. 2C), SiC₂H⁺ (FIG. 2D), SiC₂H₃⁺ (FIG. 2E), SiC₃H₉⁺ (FIG. 2F), and SiO⁺ (FIG. 2G).

FIGS. 3A to 3E show the results of example 1 of the herein described TOF-SIMS measurement (negative ions) displaying the species C₄⁻ (FIG. 3A), C₄H₃⁻ (FIG. 3B), SiC⁻ (FIG. 3C), SiCH₃O⁻ (FIG. 3D) and SiO₃⁻ (FIG. 3E).

### DETAILED DESCRIPTION

In one aspect, this disclosure provides a coated element having a surface, wherein at least a part of the surface of the element is coated by a coating, wherein the coating comprises, in this order:
a smoothing layer (SL);
a barrier layer (BL); and
a protection layer (PL),
   wherein the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1*10⁵, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and/or
   wherein the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1*10⁵, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and/or
   wherein the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10¹⁰, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS,
   wherein the coated element optionally has a gliding force of 0.1 to 10.0 N, and/or a breaking force of 1 N to 18 N.

In one embodiment of the coated element, the coating comprises, in this order:
a smoothing layer (SL);
a barrier layer (BL); and
a protection layer (PL),
   wherein the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and/or
   wherein the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and/or
   wherein the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS,
   wherein the coated element optionally has a gliding force of 0.1 to 10.0 N, and/or a breaking force of 1 N to 18 N.

In one embodiment of the coated element, the coating comprises, in this order:
a smoothing layer (SL);
a barrier layer (BL); and
a protection layer (PL),
   wherein the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and/or
   wherein the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and/or
   wherein the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS,
   wherein the coated element optionally has a gliding force of 0.1 to 10.0 N, and/or a breaking force of 1 N to 18 N,
   wherein the thickness of the smoothing layer is 150 nm to 1500 nm, wherein the thickness of the barrier layer is 5 nm to 500 nm, and wherein the thickness of the protection layer is 20 nm to 1000 nm.

In one embodiment of the coated element, the coating comprises, in this order:
a smoothing layer (SL);
a barrier layer (BL); and
a protection layer (PL),
   wherein the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and
   wherein the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and
   wherein the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS,
   wherein the coated element optionally has a gliding force of 0.1 to 10.0 N, and/or a breaking force of 1 N to 18 N,
   wherein the thickness of the smoothing layer is 150 nm to 1500 nm, and/or wherein the thickness of the barrier layer is 5 nm to 500 nm, and/or wherein the thickness of the protection layer is 20 nm to 1000 nm.

In a further aspect, this disclosure provides a coated element having a surface, wherein at least a part of the surface of the element is coated by a coating, wherein the coating comprises, in this order:
a smoothing layer (SL);
a barrier layer (BL); and
a protection layer (PL),
   wherein the smoothing layer has a thickness of 1 nm or more, and/or
   wherein the barrier layer has a thickness of 1 nm or more, and/or
   wherein the protection layer has a gliding force of 0.1 to 10.0 N, and/or a breaking force of 1 N to 18 N, and/or
   wherein the coated element has a gliding force of 0.1 to 10.0 N, and/or a breaking force of 1 N to 18 N.

In one embodiment, the coated element comprises, in this order:
a smoothing layer (SL);
a barrier layer (BL); and
a protection layer (PL),
   wherein the smoothing layer has a thickness of 100 nm to 2000 nm, and/or
   wherein the barrier layer has a thickness of 5 nm to 500 nm, and/or
   wherein the protection layer has a gliding force of 0.1 to 10.0 N, and/or a breaking force of 1 N to 18 N, and/or
   wherein the coated element has a gliding force of 0.1 to 10.0 N, and/or a breaking force of 1 N to 18 N.

In one embodiment, the coated element comprises, in this order:
a smoothing layer (SL);
a barrier layer (BL); and
a protection layer (PL),
   wherein the smoothing layer has a thickness of 100 nm to 2000 nm, wherein the barrier layer has a thickness of 5 nm to 500 nm, and wherein the thickness of the protection layer is 20 nm to 1000 nm,
   wherein the protection layer has a gliding force of 0.1 to 10.0 N, and/or a breaking force of 1 N to 18 N, and/or
   wherein the coated element has a gliding force of 0.1 to 10.0 N, and/or a breaking force of 1 N to 18 N.

In one embodiment, the coated element comprises, in this order:
a smoothing layer (SL);
a barrier layer (BL); and
a protection layer (PL),
   wherein the smoothing layer has a thickness of 100 nm to 2000 nm, and/or wherein the barrier layer has a thickness of 5 nm to 500 nm, and/or wherein the thickness of the protection layer is 20 nm to 1000 nm,
   wherein the protection layer has a gliding force of 0.1 to 10.0 N, and a breaking force of 1 N to 18 N, and/or wherein the coated element has a gliding force of 0.1 to 10.0 N, and a breaking force of 1 N to 18 N.

In one embodiment, the coated element further comprising a gliding layer (GL),
wherein the gliding layer is adjacent to the protection layer,
wherein the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10¹⁰, wherein [SiC₃H₉⁺]_{GL} are the counts of [SiC₃H₉⁺] ions in the gliding layer, measured by a TOF-SIMS; and wherein [Si₂O⁺]_{GL} are the counts of [Si₂O⁺] ions in the gliding layer, measured by a TOF-SIMS.
   In one embodiment of the coated element comprising the smoothing layer (SL), the barrier layer (BL), the protection layer (PL) and the gliding layer (GL),
the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and/or
the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and/or
the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS, and/or
the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10⁵, wherein [SiC₃H₉⁺]_{GL} are the counts of [SiC₃H₉⁺] ions in the gliding layer, measured by a TOF-SIMS; and wherein [Si₂O⁺]_{GL} are the counts of [Si₂O⁺] ions in the gliding layer, measured by a TOF-SIMS.

In one embodiment of the coated element comprising the smoothing layer (SL), the barrier layer (BL), the protection layer (PL) and the gliding layer (GL),
the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, and/or
the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, and/or
the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, and/or
the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10⁵,
wherein the smoothing layer has a thickness of 100 nm to 2000 nm, wherein the barrier layer has a thickness of 5 nm to 500 nm, wherein the protection layer has a thickness of 20 nm to 1000 nm, and wherein the gliding layer has a thickness of 30 nm to 1000 nm.

In one embodiment of the coated element comprising the smoothing layer (SL), the barrier layer (BL), the protection layer (PL) and the gliding layer (GL),
the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, and
the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, and
the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, and
the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10⁵,
wherein the smoothing layer has a thickness of 100 nm to 2000 nm, and/or wherein the barrier layer has a thickness of 5 nm to 500 nm, and/or wherein the protection layer has a thickness of 20 nm to 1000 nm, and/or wherein the gliding layer has a thickness of 30 nm to 1000 nm.

In one embodiment of the coated element comprising the smoothing layer (SL), the barrier layer (BL), the protection layer (PL) and the gliding layer (GL),
the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, and/or
the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, and/or
the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, and/or
the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10⁵,
wherein the smoothing layer has a thickness of 100 nm to 2000 nm, wherein the barrier layer has a thickness of 5 nm to 500 nm, wherein the protection layer has a thickness of 20 nm to 1000 nm, and wherein the gliding layer has a thickness of 30 nm to 1000 nm,
wherein the coefficients for both static and gliding friction are less than 10 N.

In one embodiment of the coated element comprising the smoothing layer (SL), the barrier layer (BL), the protection layer (PL) and the gliding layer (GL),
the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, and/or
the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, and/or
the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, and/or
the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10⁵,
wherein the smoothing layer has a thickness of 100 nm to 2000 nm, wherein the barrier layer has a thickness of 5 nm to 500 nm, wherein the protection layer has a thickness of 20 nm to 1000 nm, and wherein the gliding layer has a thickness of 30 nm to 1000 nm, wherein the coefficients for both static and gliding friction are between 1 N and 10 N.

In one embodiment, the coated element comprises, preferably consists of glass or polymer, preferably polymer, more preferably cyclic olefin copolymer (COC) and/or cyclic olefin polymer (COP) and/or polypropylene (PP), more preferably cyclic olefin copolymer (COC).

In a most preferred embodiment, the coated element consists of cyclic olefin copolymer (COC) or cyclic olefin polymer (COP).

In one embodiment of the coated element, the gliding layer satisfies one or more of the following parameters:
a1_{GL}≤[SiC₃H₉⁺]_{GL}/[Si₂O⁺]_{GL}≤a2_{GL}, wherein a1_{GL} is 1.1, wherein a2_{GL} is 1*10¹⁰, wherein [SiC₃H₉⁺]_{GL} are the counts of [SiC₃H₉⁺] ions in the gliding layer, measured by a TOF-SIMS; and wherein [Si₂O⁺]_{GL} are the counts of [Si₂O⁺] ions in the gliding layer, measured by a TOF-SIMS, and
b1_{GL}≤[SiC₂H⁺]_{GL}/[SiO⁺]_{GL}≤b2_{GL}, wherein b1_{GL} is 1.1, wherein b2_{GL} is 1000, wherein [SiC₂H⁺]_{GL} are the counts of [SiC₂H⁺] ions in the gliding layer, measured by a TOF-SIMS; and wherein [SiO⁺]_{GL} are the counts of [SiO⁺] ions in the gliding layer, measured by a TOF-SIMS.

In one embodiment of the coated element, the gliding layer satisfies one or more of the following parameters:
a1_{GL}≤[SiC₃H₉⁺]_{GL}/[Si₂O⁺]_{GL}≤a2_{GL}, wherein a1_{GL} is 1.1, wherein a2_{GL} is 1*10⁵, wherein [SiC₃H₉⁺]_{GL} are the counts of [SiC₃H₉⁺] ions in the gliding layer, measured by a TOF-SIMS; and wherein [Si₂O⁺]_{GL} are the counts of [Si₂O⁺] ions in the gliding layer, measured by a TOF-SIMS, and
b1_{GL}≤[SiC₂H⁺]_{GL}/[SiO⁺]_{GL}≤b2_{GL}, wherein b1_{GL} is 1.1, wherein b2_{GL} is 1000, wherein [SiC₂H⁺]_{GL} are the counts of [SiC₂H⁺] ions in the gliding layer, measured by a TOF-SIMS; and wherein [SiO⁺]_{GL} are the counts of [SiO⁺] ions in the gliding layer, measured by a TOF-SIMS.

In one embodiment of the coated element, the protection layer satisfies one or more of the following parameters:
a1_{PL}≤[Si₂O⁺]_{PL}/[SiC₃H₉⁺]_{PL}≤a2_{PL}, wherein a1_{PL} is 1.0, wherein a2_{PL} is 1*10¹⁰, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS, and
b1_{PL}≤[SiC₂H⁺]_{PL}/[SiO⁺]_{PL}≤b2_{PL}, wherein b1_{PL} is 0.5, wherein b2_{PL} is 1*10⁵, wherein [SiC₂H⁺]_{PL} are the counts of [SiC₂H⁺] ions in the protection layer, measured by a TOF-SIMS; and wherein [SiO⁺]_{PL} are the counts of [SiO⁺] ions in the protection layer, measured by a TOF-SIMS.

In one embodiment of the coated element, the protection layer satisfies one or more of the following parameters:
a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS, and
b1_{PL}≤[SiC₂H⁺]_{PL}/[SiO⁺]_{PL}≤b2_{PL}, wherein b1_{PL} is 0.5, wherein b2_{PL} is 1*10⁵, wherein [SiC₂H⁺]_{PL} are the counts of [SiC₂H⁺] ions in the protection layer, measured by a TOF-SIMS; and wherein [SiO⁺]_{PL} are the counts of [SiO⁺] ions in the protection layer, measured by a TOF-SIMS.

In one embodiment of the coated element, the barrier layer satisfies one or more of the following parameters:
a1_{BL}≤[Si₂O⁺]_{BL}/[SiC₃H₉⁺]_{BL}≤a2_{BL}, wherein a1_{BL} is 1, wherein a2_{BL} is 1*10⁵, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and
b1_{BL}≤[SiO⁺]_{BL}/[SiC₂H⁺]_{BL}≤b2_{BL}, wherein b1_{BL} is 1, wherein b2_{BL} is 1*10⁵, wherein [SiC₂H⁺]_{BL} are the counts of [SiC₂H⁺] ions in the barrier layer, measured by a TOF-SIMS, wherein [SiO⁺]_{BL} are the counts of [SiO⁺] ions in the barrier layer, measured by a TOF-SIMS.

In one embodiment of the coated element, the barrier layer satisfies one or more of the following parameters:
a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and
b1_{BL}≤[SiO⁺]_{BL}/[SiC₂H⁺]_{BL}≤b2_{BL}, wherein b1_{BL} is 1, wherein b2_{BL} is 1*10⁵, wherein [SiC₂H⁺]_{BL} are the counts of [SiC₂H⁺] ions in the barrier layer, measured by a TOF-SIMS, wherein [SiO⁺]_{BL} are the counts of [SiO⁺] ions in the barrier layer, measured by a TOF-SIMS.

In one embodiment of the coated element, the smoothing layer satisfies one or more of the following parameters:
a1_{SL}≤[Si₂O⁺]_{SL}/[SiC₃H₉⁺]_{SL}≤a2_{SL}, wherein a1_{SL} is 1.0, wherein a2_{SL} is 1*10⁵, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and
b1_{SL}≤[SiC₂H⁺]_{SL}/[SiO⁺]_{SL}≤b2_{SL}, wherein b1_{SL} is 1.0, wherein b2_{SL} is 1*10⁵, wherein [SiC₂H⁺]_{SL} are the counts of [SiC₂H⁺] ions in the smoothing layer, measured by a TOF-SIMS, wherein [SiO⁺]_{SL} are the counts of [SiO⁺] ions in the smoothing layer, measured by a TOF-SIMS.

In one embodiment of the coated element, the smoothing layer satisfies one or more of the following parameters:
a1_{SL}≤[Si₂O⁺]_{SL}/[SiC₃H₉⁺]_{SL}≤a2_{SL}, wherein a1_{SL} is 1.0, wherein a2_{SL} is 1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and
b1_{SL}≤[SiC₂H⁺]_{SL}/[SiO⁺]_{SL}≤b2_{SL}, wherein b1_{SL} is 1.0, wherein b2_{SL} is 1*10⁵, wherein [SiC₂H⁺]_{SL} are the counts of [SiC₂H⁺] ions in the smoothing layer, measured by a TOF-SIMS, wherein [SiO⁺]_{SL} are the counts of [SiO⁺] ions in the smoothing layer, measured by a TOF-SIMS.

In one embodiment of the coated element, the thickness of the smoothing layer is 1 nm to 25000 nm, or 2 nm to 10000 nm, or 5 nm to 5000 nm, or 100 nm to 2000 nm, or 150 nm to 1500 nm, or 200 nm to 1000 nm, or 250 nm to 500 nm.

In one embodiment of the coated element, the time a sputter gun needs to reach the surface of the element is 0.5 minutes to 150 minutes, or 30 to 120 minutes, or 50 to 100 minutes; and/or wherein in the case positive ions are measured, the point, when the sputter gun reaches the surface of the element is the point, when the counts of [Si₂O⁺] ions are equal to the counts of [C₃H₅⁺] ions; and/or wherein in the case negative ions are measured, the point, when the sputter gun reaches the surface of the element is the point, when the counts of [C₄H₃⁻] ions are equal to the counts of [SiO₃⁻] ions; and/or wherein the counts of the ions, preferably the positive and negative ions are obtainable by the method described in the description.

In one embodiment of the coated element, the element is a pharmaceutical container comprising an inner surface, wherein the inner surface is coated, wherein the smoothing layer is in direct contact with the coated element, wherein the gliding layer, if present, is the outermost layer.

In one embodiment of the coated element, one or more of the following conditions is/are fulfilled:

$${[ {SiO}_{3}^{-} ]}_{GL} < {[ {SiO}_{3}^{-} ]}_{BL} ;$$

$${[ {SiO}_{3}^{-} ]}_{PL} < {[ {SiO}_{3}^{-} ]}_{BL} ;$$

$${[ {SiO}_{3}^{-} ]}_{SL} < {[ {SiO}_{3}^{-} ]}_{BL} ; and / or$$

$${[ {SiO}_{3}^{-} ]}_{GL} < {[ {SiO}_{3}^{-} ]}_{PL} ;$$

and
wherein [SiO₃⁻]_{GL} are the counts of [SiO₃⁻] ions in the gliding layer, measured by a TOF-SIMS;
wherein [SiO₃⁻]_{PL} are the counts of [SiO₃⁻] ions in the protection layer, measured by a TOF-SIMS;
wherein [SiO₃⁻]_{BL} are the counts of [SiO₃⁻] ions in the barrier layer, measured by a TOF-SIMS;
wherein [SiO₃⁻]_{SL} are the counts of [SiO₃⁻] ions in the smoothing layer, measured by a TOF-SIMS.

In one aspect, this disclosure provides a system or kit comprising the coated element according to this disclosure, wherein the element is a syringe or cartridge; a plunger, preferably a plunger rod; and optionally a closure, preferably a tip cap and/or a needle shield.

In one aspect, this disclosure provides a closed system comprising the coated element according to this disclosure, wherein the element is a syringe or cartridge; a plunger, preferably a plunger rod; and a closure, preferably a tip cap and/or a needle shield, wherein the closed coated element passes the container closure integrity test according to DIN EN ISO 8871-5:2016; chapter 4.4 in combination with Annex D.

In one embodiment, the closed system comprises a composition, preferably a pharmaceutical composition, preferably a composition containing biologics or mRNA.

In one aspect, this disclosure provides a method for coating an element, preferably a coated element according to this disclosure, comprising the steps:
(1) providing an element comprising a surface;
(2) applying a smoothing layer;
(3) applying a barrier layer;
(4) applying a protection layer; and
(5) optionally applying a gliding layer,
wherein each of the steps (2) to (4) comprises surrounding at least part of the surface of the element with a precursor, and
wherein each of the steps (2) to (4) comprises irradiating the precursor to generate a plasma using a PICVD method,
wherein the step (3) comprises establishing a flow rate of O₂ of 10 sccm or more during the irradiation of the precursor.

### Gliding Layer

In one embodiment of the coated element, the gliding layer fulfills the following parameter:

$$a {1}_{GL} \leq {[ {SiC}_{3} {H}_{9}^{+} ]}_{GL} / {[ {Si}_{2} {O}^{+} ]}_{GL} \leq a {2}_{GL} ;$$

wherein a1_{GL} is 1.1, preferably 2, more preferably 5, more preferably 10, more preferably 20, more preferably 50, more preferably 100, more preferably 200, more preferably 300, more preferably 400;
wherein a2_{GL} is 1*10¹⁰, preferably 1*10⁵, more preferably 1000, more preferably 800, more preferably 600, more preferably 500;
wherein [SiC₃H₉⁺]_{GL} are the counts of [SiC₃H₉⁺] ions in the gliding layer, measured by a TOF-SIMS; and
wherein [Si₂O⁺]_{GL} are the counts of [Si₂O⁺] ions in the gliding layer, measured by a TOF-SIMS.

In one embodiment, a1_{GL} is 1.1, preferably 2, more preferably 5, more preferably 10, more preferably 20, more preferably 50, more preferably 100, more preferably 200, more preferably 300, more preferably 400. In one embodiment, a2_{GL} is 1*10¹⁰, preferably 1*10⁵, more preferably 1000, more preferably 800, more preferably 600, more preferably 500. In one embodiment, a1_{GL} is 1.1, and a2_{GL} is 1*10¹⁰. In one embodiment, a1_{GL} is 2, and a2_{GL} is 1*10⁵. In one embodiment, a1_{GL} is 5, and a2_{GL} is 1000. In one embodiment, a1_{GL} is 10, and a2_{GL} is 1000.

In one embodiment of the coated element, the gliding layer fulfills the following parameter:

$$b {1}_{GL} \leq {[ {SiC}_{2} {H}^{+} ]}_{GL} / {[ {SiO}^{+} ]}_{GL} \leq b {2}_{GL} ;$$

wherein b1_{GL} is 1.1, preferably 1.2, more preferably 1.5, more preferably 1.8, more preferably 2.0, more preferably 2.3;
wherein b2_{GL} is 1000, preferably 100, more preferably 10, more preferably 5, more preferably 4, more preferably 3, more preferably 2.5;
wherein [SiC₂H⁺]_{GL} are the counts of [SiC₂H⁺] ions in the gliding layer, measured by a TOF-SIMS; and
wherein [SiO⁺]_{GL} are the counts of [SiO⁺] ions in the gliding layer, measured by a TOF-SIMS.

In one embodiment, b1_{GL} is 1.1, preferably 1.2, more preferably 1.5, more preferably 1.8, more preferably 2.0, more preferably 2.3. In one embodiment, b2_{GL} is 1000, preferably 100, more preferably 10, more preferably 5, more preferably 4, more preferably 3, more preferably 2.5. In one embodiment, b1_{GL} is 1.1, and b2_{GL} is 1000. In one embodiment, b1_{GL} is 2.0, and b2_{GL} is 100. In one embodiment, b1_{GL} is 2.3, and b2_{GL} is 2.5.

In one embodiment of the coated element, the gliding layer fulfills the following parameter:

$$c {1}_{GL} \leq {[ {SiC}_{3} {H}_{9}^{+} ]}_{GL} / {[ {SiO}^{+} ]}_{GL} \leq c {1}_{GL} ;$$

wherein c1_{GL} is 1.1, preferably 2, more preferably 5, more preferably 10, more preferably 20, more preferably 50, more preferably 100, more preferably 200, more preferably 300, more preferably 400;
wherein c2_{GL} is 1*10¹⁰, preferably 1*10⁵, more preferably 1000, more preferably 800, more preferably 600, more preferably 500;
wherein [SiC₃H₉⁺]_{GL} are the counts of [SiC₃H₉⁺] ions in the gliding layer, measured by a TOF-SIMS; and
wherein [SiO⁺]_{GL} are the counts of [SiO⁺] ions in the gliding layer, measured by a TOF-SIMS.

In one embodiment, c1_{GL} is 1.1, preferably 2, more preferably 5, more preferably 10, more preferably 20, more preferably 50, more preferably 100, more preferably 200, more preferably 300, more preferably 400. In one embodiment, c2_{GL} is 1*10¹⁰, preferably 1*10⁵, more preferably 1000, more preferably 800, more preferably 600, more preferably 500. In one embodiment, c1_{GL} is 1.1, and c2_{GL} is 1*10¹⁰. In one embodiment, c1_{GL} is 2, and c2_{GL} is 1*10⁵. In one embodiment, c1_{GL} is 5, and c2_{GL} is 1000. In one embodiment, c1_{GL} is 400, and c2_{GL} is 500.

In one embodiment of the coated element, the gliding layer fulfills the following parameter:

$$d {1}_{GL} \leq {[ {SiCH}_{3} {O}^{-} ]}_{GL} / {[ {SiO}_{3}^{-} ]}_{GL} \leq d {1}_{GL} ;$$

wherein d1_{GL} is 1.0, preferably 1.1, more preferably 1.5, more preferably 2, more preferably 3, more preferably 5, more preferably 8, more preferably 10, more preferably 15, more preferably 20;
wherein d2_{GL} is 1*10¹⁰, preferably 1*10⁵, more preferably 1000, more preferably 200, more preferably 100, more preferably 50, more preferably 30;
wherein [SiCH₃O⁻]_{GL} are the counts of [SiCH₃O⁻] ions in the gliding layer, measured by a TOF-SIMS; and
wherein [SiO₃⁻]_{GL} are the counts of [SiO₃⁻] ions in the gliding layer, measured by a TOF-SIMS.

In one embodiment, d1_{GL} is 1.0, preferably 1.1, more preferably 1.5, more preferably 2, more preferably 3, more preferably 5, more preferably 8, more preferably 10, more preferably 15, more preferably 20. In one embodiment, d2_{GL} is 1*10¹⁰, preferably 1*10⁵, more preferably 1000, more preferably 200, more preferably 100, more preferably 50, more preferably 30. In one embodiment, d1_{GL} is 1.0, and d2_{GL} is 1*10¹⁰. In one embodiment, d1_{GL} is 2, and d2_{GL} is 1*10⁵. In one embodiment, d1_{GL} is 5, and d2_{GL} is 1*1000. In one embodiment, d1_{GL} is 20, and d2_{GL} is 30.

In one embodiment of the coated element, the gliding layer fulfills the following parameter:

$$e {1}_{GL} \leq {[ {SiCH}_{3} {O}_{2}^{-} ]}_{GL} / {[ {SiO}_{3}^{-} ]}_{GL} \leq e {1}_{GL} ;$$

wherein e1_{GL} is 1.0, preferably 1.1, more preferably 1.5, more preferably 2, more preferably 3, more preferably 5, more preferably 8, more preferably 10, more preferably 15, more preferably 20;
wherein e2_{GL} is 1*10¹⁰, preferably 1*10⁵, more preferably 1000, more preferably 200, more preferably 100, more preferably 50, more preferably 30;
wherein [SiCH₃O₂⁻]_{GL} are the counts of [SiCH₃O₂⁻] ions in the gliding layer, measured by a TOF-SIMS; and
wherein [SiO₃⁻]_{GL} are the counts of [SiO₃⁻] ions in the gliding layer, measured by a TOF-SIMS.

In one embodiment, e1_{GL} is 1.0, preferably 1.1, more preferably 1.5, more preferably 2, more preferably 3, more preferably 5, more preferably 8, more preferably 10, more preferably 15, more preferably 20. In one embodiment, e2_{GL} is 1*10¹⁰, preferably 1*10⁵, more preferably 1000, more preferably 200, more preferably 100, more preferably 50, more preferably 30. In one embodiment, e1_{GL} is 1.0, and e2_{GL} is 1*10¹⁰. In one embodiment, e1_{GL} is 2, and e2_{GL} is 1*10⁵. In one embodiment, e1_{GL} is 5, and e2_{GL} is 1000. In one embodiment, e1_{GL} is 20, and e2_{GL} is 30.

In one embodiment of the coated element, the thickness of the gliding layer is 10 nm to 10000 nm, or 15 nm to 5000 nm, or 20 nm to 2000 nm, or 30 nm to 1000 nm, or 40 nm to 500 nm, or 50 nm to 200 nm.

### Protection Layer

In one embodiment of the coated element, the protection layer comprises silicone, preferably:
i) linear silicone; preferably linear polydimethylsiloxane, more preferably hexamethyldisiloxane (HMDSO), octamethyltrisiloxane, decamethyltetrasiloxane; and/or
ii) cyclic silicone; preferably octamethylcyclotetrasiloxane, decamethylcyclopentasiloxane, dodecamethylcyclohexasiloxane, tetramethylcyclotetrasiloxane, pentamethylcyclopentasiloxane; and/or
iii) branched siloxane; and/or
iv) a copolymer comprising any of i) to iii).
   In one embodiment of the coated element, the protection layer fulfills the following parameter:

$$a {1}_{PL} \leq {[ {Si}_{2} {O}^{+} ]}_{PL} / {[ {SiC}_{3} {H}_{9}^{+} ]}_{PL} \leq a {2}_{PL} ;$$

wherein a1_{PL} is 1.0, preferably 1.1, more preferably 1.2, more preferably 1.3, more preferably 1.4, more preferably 1.5, more preferably 2.0, more preferably 3.0;
wherein a2_{PL} is 1*10¹⁰, preferably 1*10⁵, more preferably 100, more preferably 20, more preferably 15, more preferably 10, more preferably 7;
wherein [SiC₃H₉⁺]_{PL} are the counts of [SiC₃H₉⁺] ions in the protection layer, measured by a TOF-SIMS; and
wherein [Si₂O⁺]_{PL} are the counts of [Si₂O⁺] ions in the protection layer, measured by a TOF-SIMS.

In one embodiment, a1_{PL} is 1.0, preferably 1.1, more preferably 1.2, more preferably 1.3, more preferably 1.4, more preferably 1.5, more preferably 2.0, more preferably 3.0. In one embodiment, a2_{PL} is 1*10¹⁰, preferably 1*10⁵, more preferably 100, more preferably 20, more preferably 15, more preferably 10, more preferably 7. In one embodiment, a1_{PL} is 1.0, and a2_{PL} is 1*10¹⁰. In one embodiment, a1_{PL} is 2.0, and a2_{PL} is 1*10⁵. In one embodiment, a1_{PL} is 2.0, and a2_{PL} is 100. In one embodiment, a1_{PL} is 3.0, and a2_{PL} is 7.

In one embodiment of the coated element, the protection layer fulfills the following parameter:

$$b {1}_{PL} \leq {[ {SiC}_{2} {H}^{+} ]}_{PL} / {[ {SiO}^{+} ]}_{PL} \leq b {2}_{PL} ;$$

wherein [SiC₂H⁺]_{PL} are the counts of [SiC₂H⁺] ions in the protection layer, measured by a TOF-SIMS; and
wherein [SiO⁺]_{PL} are the counts of [SiO⁺] ions in the protection layer, measured by a TOF-SIMS; and
wherein b1_{PL} is 0.5, preferably 0.8, more preferably 1.0, more preferably 1.1, more preferably 1.2; and
wherein b2_{PL} is 1*10⁵, preferably 100, more preferably 25, more preferably 15, more preferably 5, more preferably 2, more preferably 1.5.

In one embodiment, b1_{PL} is 0.5, preferably 0.8, more preferably 1.0, more preferably 1.1, more preferably 1.2. In one embodiment, b2_{PL} is 1*10⁵, preferably 100, more preferably 25, more preferably 15, more preferably 5, more preferably 2, more preferably 1.5. In one embodiment, b1_{PL} is 0.5, and b2_{PL} is 1*10⁵. In one embodiment, b1_{PL} is 0.8, and b2_{PL} is 100. In one embodiment, b1_{PL} is 1.2, and b2_{PL} is 1.5.

In one embodiment of the coated element, the protection layer fulfills the following parameter:

$$c {1}_{PL} \leq {[ {SiO}^{+} ]}_{PL} / {[ {SiC}_{3} {H}_{9}^{+} ]}_{PL} \leq c {1}_{PL} ;$$

wherein c1_{PL} is 0.5, preferably 0.8, more preferably 1.0, more preferably 1.5, more preferably 2.0, more preferably 2.0, more preferably 2.1;
wherein c2_{PL} is 1*10⁵, preferably 100, more preferably 25, more preferably 20, more preferably 15, more preferably 4, more preferably 2.5;
wherein [SiC₃H₉⁺]_{PL} are the counts of [SiC₃H₉⁺] ions in the protection layer, measured by a TOF-SIMS; and
wherein [SiO⁺]_{PL} are the counts of [SiO⁺] ions in the protection layer, measured by a TOF-SIMS.

In one embodiment, c1_{PL} is 0.5, preferably 0.8, more preferably 1.0, more preferably 1.5, more preferably 2.0, more preferably 2.0, more preferably 2.1. In one embodiment, c2_{PL} is 1*10⁵, preferably 100, more preferably 25, more preferably 20, more preferably 15, more preferably 4, more preferably 2.5. In one embodiment, c1_{PL} is 2.1, and c2_{PL} is 2.5. In one embodiment, c1_{PL} is 1.0, and c2_{PL} is 100. In one embodiment, c1_{PL} is 0.5, and c2_{PL} is 1*10⁵.

In one embodiment of the coated element, the thickness of the protection layer is 1 nm to 8000 nm, or 5 nm to 5000 nm, or 10 nm to 2000 nm, or 20 nm to 1000 nm, or 30 nm to 500 nm, or 50 nm to 300 nm.

### Barrier Layer

In one embodiment of the coated element, the barrier layer fulfills the following parameter:

$$a {1}_{BL} \leq {[ {Si}_{2} {O}^{+} ]}_{BL} / {[ {SiC}_{3} {H}_{9}^{+} ]}_{BL} \leq a {2}_{BL} ;$$

wherein a1_{BL} is 1, preferably 2, more preferably 4, more preferably 10, more preferably 15, more preferably 20, more preferably 25; and
wherein a2_{BL} is 1*10⁵, preferably 1000, more preferably 500, more preferably 250, more preferably 150, more preferably 100, more preferably 50;
wherein [SiC₃H₉⁺]_{BL} are the counts of [SiC₃H₉⁺] ions in the barrier layer, measured by a TOF-SIMS; and
wherein [Si₂O⁺]_{BL} are the counts of [Si₂O⁺] ions in the barrier layer, measured by a TOF-SIMS.

In one embodiment, a1_{BL} is 1, preferably 2, more preferably 4, more preferably 10, more preferably 15, more preferably 20, more preferably 25. In one embodiment, a2_{BL} is 1*10⁵, preferably 1000, more preferably 500, more preferably 250, more preferably 150, more preferably 100, more preferably 50. In one embodiment, a1_{BL} is 1, and a2_{BL} is 1*10⁵. In one embodiment, a1_{BL} is 2, and a2_{BL} is 1000. In one embodiment, a1_{BL} is 4, and a2_{BL} is 100. In one embodiment, a1_{BL} is 25, and a2_{BL} is 50.

In one embodiment of the coated element, the barrier layer fulfills the following parameter:

$$b {1}_{B L} \leq {[ {SiO}^{+} ]}_{B L} {/ [ Si {C}_{2} {H}^{+} ]}_{B L} \leq b {2}_{B L} ;$$

wherein b1_{BL} is 1, preferably 5, more preferably 10, more preferably 20, more preferably 30, more preferably 40, more preferably 45; and
wherein b2_{BL} is 1*10⁵, preferably 1000, more preferably 500, more preferably 250, more preferably 150, more preferably 100, more preferably 80;
wherein [SiC₂H⁺]_{BL} are the counts of [SiC₂H⁺] ions in the barrier layer, measured by a TOF-SIMS; and
wherein [SiO⁺]_{BL} are the counts of [SiO⁺] ions in the barrier layer, measured by a TOF-SIMS.

In one embodiment, b1_{BL} is 1, preferably 5, more preferably 10, more preferably 20, more preferably 30, more preferably 40, more preferably 45. In one embodiment, b2_{BL} is 1*10⁵, preferably 1000, more preferably 500, more preferably 250, more preferably 150, more preferably 100, more preferably 80. In one embodiment, b1_{BL} is 1, and b2_{BL} is 1*10⁵. In one embodiment, b1_{BL} is 5, and b2_{BL} is 1000. In one embodiment, b1_{BL} is 10, and b2_{BL} is 500. In one embodiment, b1_{BL} is 45, and b2_{BL} is 80.

In one embodiment of the coated element, the barrier layer fulfills the following parameter:

$$c {1}_{B L} \leq {[ {SiO}^{+} ]}_{B L} {/ [ Si {C}_{3} {H}_{9}^{+} ]}_{B L} \leq c {1}_{B L} ;$$

wherein c1_{BL} is 1, preferably 5, more preferably 10, more preferably 50, more preferably 75, more preferably 100, more preferably 110; and
wherein c2_{BL} is 1*10⁵, preferably 1000, more preferably 500, more preferably 400, more preferably 300, more preferably 200, more preferably 150;
wherein [SiC₃H₉⁺]_{BL} are the counts of [SiC₃H₉⁺] ions in the barrier layer, measured by a TOF-SIMS; and
wherein [SiO⁺]_{BL} are the counts of [SiO⁺] ions in the barrier layer, measured by a TOF-SIMS.

In one embodiment, c1_{BL} is 1, preferably 5, more preferably 10, more preferably 50, more preferably 75, more preferably 100, more preferably 110. In one embodiment, c2_{BL} is 1*10⁵, preferably 1000, more preferably 500, more preferably 400, more preferably 300, more preferably 200, more preferably 150. In one embodiment, c1_{BL} is 1, and c2_{BL} is 1*10⁵. In one embodiment, c1_{BL} is 10, and c2_{BL} is 1000. In one embodiment, c1_{BL} is 110, and c2_{BL} is 150.

In one embodiment of the coated element, the thickness of the barrier layer is 1 nm to 1000 nm, or 5 nm to 500 nm, or 10 nm to 200 nm, or 20 nm to 150 nm.

### Smoothing Layer

In one embodiment of the coated element, the smoothing layer fulfills the following parameter:

$$a {1}_{S L} \leq {[ S {i}_{2} {O}^{+} ]}_{S L} {/ [ Si {C}_{3} {H}_{9}^{+} ]}_{S L} \leq a {2}_{S L} ;$$

wherein a1_{SL} is 1.0, preferably 2.0, preferably 4.0, more preferably 6.0, more preferably 8.0, more preferably 9.0, more preferably 10.0;
wherein a2_{SL} is 1*10⁵, preferably 1000, preferably 500, more preferably 250, more preferably 100, more preferably 50, more preferably 25, more preferably 15;
wherein [SiC₃H₉⁺]_{SL} are the counts of [SiC₃H₉⁺] ions in the smoothing layer, measured by a TOF-SIMS; and
wherein [Si₂O⁺]_{SL} are the counts of [Si₂O⁺] ions in the smoothing layer, measured by a TOF-SIMS.

In one embodiment, a1_{SL} is 1.0, preferably 2.0, preferably 4.0, more preferably 6.0, more preferably 8.0, more preferably 9.0, more preferably 10.0. In one embodiment, a2_{SL} is 1*10⁵, preferably 1000, preferably 500, more preferably 250, more preferably 100, more preferably 50, more preferably 25, more preferably 15. In one embodiment, a1_{SL} is 1.0, and a2_{SL} is 1*10⁵. In one embodiment, a1_{SL} is 2.0, and a2_{SL} is 1000. In one embodiment, a1_{SL} is 6.0, and a2_{SL} is 250. In one embodiment, a1_{SL} is 10.0, and a2_{SL} is 15.

In one embodiment of the coated element, the smoothing layer fulfills the following parameter:

$$b {1}_{S L} \leq {[ Si {C}_{2} {H}^{+} ]}_{S L} {/ [ Si {O}^{+} ]}_{S L} \leq b {2}_{S L} ;$$

wherein [SiC₂H⁺]_{SL} are the counts of [SiC₂H⁺] ions in the smoothing layer, measured by a TOF-SIMS; and
wherein [SiO⁺]_{SL} are the counts of [SiO⁺] ions in the smoothing layer, measured by a TOF-SIMS; and
wherein b1_{SL} is 1.0, preferably 2.0, more preferably 2.5, more preferably 3.0, more preferably 3.5, more preferably 4.0;
wherein b2_{SL} is 1*10⁵, preferably 100, more preferably 25, more preferably 20, more preferably 15, more preferably 9, more preferably 7.

In one embodiment, b1_{SL} is 1.0, preferably 2.0, more preferably 2.5, more preferably 3.0, more preferably 3.5, more preferably 4.0. In one embodiment, b2_{SL} is 1*10⁵, preferably 100, more preferably 25, more preferably 20, more preferably 15, more preferably 9, more preferably 7. In one embodiment, b1_{SL} is 1.0, and b2_{SL} is 1*10⁵. In one embodiment, b1_{SL} is 2.0, and b2_{SL} is 100. In one embodiment, b1_{SL} is 3.0, and b2_{SL} is 25. In one embodiment, b1_{SL} is 4.0, and b2_{SL} is 7.

In one embodiment of the coated element, the smoothing layer fulfills the following parameter:

$$c {1}_{S L} \leq {[ {SiO}^{+} ]}_{S L} {/ [ Si {C}_{3} {H}_{9}^{+} ]}_{S L} \leq c {1}_{S L} ;$$

wherein c1_{SL} is 0.5, preferably 0.8, more preferably 1.0, more preferably 1.5, more preferably 2.0, more preferably 2.0, more preferably 2.1;
wherein c2_{PL} 1*10⁵, preferably 100, more preferably 25, more preferably 20, more preferably 15, more preferably 4, more preferably 2.5;
wherein [SiC₃H₉⁺]_{SL} are the counts of [SiC₃H₉⁺] ions in the smoothing layer, measured by a TOF-SIMS; and
wherein [SiO⁺]_{SL} are the counts of [SiO⁺] ions in the smoothing layer, measured by a TOF-SIMS.

In one embodiment, c1_{SL} is 0.5, preferably 0.8, more preferably 1.0, more preferably 1.5, more preferably 2.0, more preferably 2.0, more preferably 2.1. In one embodiment, c2_{PL} 1*10⁵, preferably 100, more preferably 25, more preferably 20, more preferably 15, more preferably 4, more preferably 2.5. In one embodiment, c1_{SL} is 0.5, and c2_{PL} 1*10⁵. In one embodiment, c1_{SL} is 1.0, and c2_{PL} 100. In one embodiment, c1_{SL} is 2.1, and c2_{PL} 2.5.

In one embodiment of the coated element, the smoothing layer fulfills the following parameter:

$$x {1}_{S L} \leq {[ Si {C}_{2} {H}^{+} ]}_{S L} {/ [ {C}_{3} {H}_{5}^{+} ]}_{S L} \leq x {2}_{S L} ;$$

wherein [SiC₂H⁺]_{SL} are the counts of [SiC₂H⁺] ions in the smoothing layer, measured by a TOF-SIMS; and
wherein [C₃H₅⁺]_{SL} are the counts of [C₃H₅⁺] ions in the smoothing layer, measured by a TOF-SIMS; and
wherein x1_{SL} is 1, preferably 2, more preferably 10, more preferably 50, more preferably 100;
and wherein x2_{SL} is 1*10¹⁰, preferably 1*10⁵, more preferably 1*10⁴, more preferably 1*10³.

In one embodiment, x1_{SL} is 1, preferably 2, more preferably 10, more preferably 50, more preferably 100. In one embodiment, x2_{SL} is 1*10¹⁰, preferably 1*10⁵, more preferably 1*10⁴, more preferably 1*10³. In one embodiment, x1_{SL} is 1, and x2_{SL} is 1*10¹⁰. In one embodiment, x1_{SL} is 10, and x2_{SL} is 1*10⁵. In one embodiment, x1_{SL} is 100, and x2_{SL} is 1*10³.

In one embodiment of the coated element, the smoothing layer fulfills the following parameter(s):

$$y {1}_{S L} \leq {[ S {i}_{2} {O}^{+} ]}_{S L} {/ [ {C}_{3} {H}_{5}^{+} ]}_{S L} \leq y {2}_{S L} ;$$

wherein [Si₂O⁺]_{SL} are the counts of [Si₂O⁺] ions in the smoothing layer, measured by a TOF-SIMS;
wherein [C₃H₅⁺]_{SL} are the counts of [C₃H₅⁺] ions in the smoothing layer, measured by a TOF-SIMS;
wherein y1_{SL} is 1; preferably 5, more preferably 20, more preferably 50, more preferably 100; and
wherein y2_{SL} is 1*10¹⁰, preferably 1*10⁵, more preferably 1*10⁴, more preferably 1*10³.

In one embodiment, y1_{SL} is 1; preferably 5, more preferably 20, more preferably 50, more preferably 100. In one embodiment, y2_{SL} is 1*10¹⁰, preferably 1*10⁵, more preferably 1*10⁴, more preferably 1*10³. In one embodiment, y1_{SL} is 1, and y2_{SL} is 1*10¹⁰. In one embodiment, y1_{SL} is 5, and y2_{SL} is 1*10⁵. In one embodiment, y1_{SL} is 100, and y2_{SL} is 1*10³.

In one embodiment of the coated element, the smoothing layer fulfills the following pa-rameter(s):

$$z {1}_{S L} \leq {[ {SiO}^{+} ]}_{S L} {/ [ {C}_{3} {H}_{5}^{+} ]}_{S L} \leq z {2}_{S L} ;$$

wherein [SiO⁺]_{SL} are the counts of [SiO⁺] ions in the smoothing layer, measured by a TOF-SIMS;
wherein [C₃H₅⁺]_{SL} are the counts of [C₃H₅⁺] ions in the smoothing layer, measured by a TOF-SIMS;
wherein z1_{SL} is 1; preferably 5, more preferably 20, more preferably 50, more preferably 70; and
wherein z2_{SL} is 1*10¹⁰, preferably 1*10⁵, more preferably 1*10⁴, more preferably 1*10³.

In one embodiment of the coated element, the thickness of the smoothing layer is 1 nm to 20000 nm, preferably 5 nm to 5000, more preferably 100 nm to 2000 nm, more preferably 150 nm to 1500 nm, more preferably 200 nm to 1000 nm, more preferably 250 nm to 500 nm.

In one embodiment, z1_{SL} is 1; preferably 5, more preferably 20, more preferably 50, more preferably 70. In one embodiment, z2_{SL} is 1*10¹⁰, preferably 1*10⁵, more preferably 1*10⁴, more preferably 1*10³. In one embodiment, z1_{SL} is 1, and z2_{SL} is 1*10¹⁰. In one embodiment, z1_{SL} is 5, and z2_{SL} is 1*10⁵. In one embodiment, z1_{SL} is 70, and z2_{SL} is 1*10³.

### Coated Element

In one embodiment of the coated element, one or both of following equations is fulfilled:

$$\begin{matrix}R 3 \leq {t}_{S L} / {t}_{C O} ; and / or \\ {t}_{S L} / {t}_{C O} \leq R 4 ;\end{matrix}$$

wherein t_{SL} is the time sputter through the smoothing layer; and
wherein t_{CO} is the time sputter through the coating;
wherein R3 is 0.01, preferably 0.05, more preferably 0.10, more preferably 0.20, more preferably 0.30, more preferably 0.40, more preferably 0.50, more preferably 0.60, more preferably 0.65; and
wherein R4 is 5.00, preferably 4.00, more preferably 3.00, more preferably 2.00, more preferably 1.00, more preferably 0.80, more preferably 0.70.

In one embodiment of the coated element, the time a sputter gun needs to reach the surface of the element is 0.5 minutes to 150 minutes, preferably 30 to 120 minutes, more preferably 50 to 100 minutes; and/or
wherein in the case positive ions are measured, the point, when the sputter gun reaches the surface of the element is the point, when the counts of [Si₂O⁺] ions are equal to the counts of [C₃H₅⁺] ions; and/or
wherein in the case negative ions are measured, the point, when the sputter gun reaches the surface of the element is the point, when the counts of [C₄H₃⁻] ions are equal to the counts of [SiO₃⁻] ions; and/or
wherein the counts of the ions, preferably the positive and negative ions are obtainable by the method described in the description.

In one embodiment of the coated element, the at least a part of the element is 10% to 90%, preferably 20% to 80%, more preferably 30% to 70%, more preferably 40% to 60% of the surface of the element.

In one embodiment of the coated element, the element is a pharmaceutical container comprising an inner surface, and
wherein the at least a part of the surface comprises at least a part of the inner surface of the pharmaceutical container, preferably comprises the inner surface of the pharmaceutical container, more preferably is the inner surface of the pharmaceutical container.

In one embodiment of the coated element, the smoothing layer is in direct contact with the element.

In one embodiment of the coated element, the gliding layer is the outermost layer.

In one embodiment of the coated element, the element comprises a partially uncoated surface, and
wherein one or both of following equations is fulfilled:

$$\begin{matrix}R 1 \leq {T}_{S L} / SRz ; and / or \\ {T}_{S L} / SRz \leq R 2 ;\end{matrix}$$

wherein R1 [nm/nm] is 0.005, 0.01, or 0.05, or 0.1, preferably 0.2, more preferably 0.3, more preferably 0.5, more preferably 1.0, more preferably 2.0, more preferably 3.0, more preferably 4.0, more preferably 5.0; and/or
wherein R2 [nm/nm] is 20.0, preferably 18.0, more preferably 15.0, more preferably 12.0, more preferably 10.0, more preferably 8.0, more preferably 6.0;
wherein T_{SL} is the thickness of the smoothing layer in at least a part of the coating; and
wherein SRz is the surface roughness depth of at least a part of the partially uncoated surface of the element.

In one embodiment of the coated element, the roughness SRz of the partially uncoated surface of the element is 1 nm to 5000 nm, 10 nm to 3000 nm, 50 nm to 2000 nm, 70 nm to 1000 nm, or 100 nm to 500 nm. In one embodiment of the coated element, the roughness SRz of the partially uncoated surface of the element is 1 nm or more, 10 nm or more, 50 nm or more, 70 nm or more, or 100 nm or more. In one embodiment of the coated element, the roughness SRz of the partially uncoated surface of the element is 5000 nm or less, 3000 nm or less, 2000 nm or less, 1000 nm or less, or 500 nm or less.

In one embodiment of the coated element, the gliding layer, the protection layer, the barrier layer and/or the smoothing layer comprise(s) SiOₓC_{y}, measured by XPS,
wherein x is from 0.3 to 3.0, preferably 0.5 to 2.0, more preferably 0.9 to 1.5, and/or
wherein y is from 0 to 6.0, preferably 0.5 to 4.0, more preferably 2.5 to 3.5.

In one embodiment of the coated element, one or more of the following conditions is/are fulfilled:

$$\begin{matrix}{[ {SiO}_{3}^{-} ]}_{G L} < {[ {SiO}_{3}^{-} ]}_{B L} ; \\ {[ {SiO}_{3}^{-} ]}_{PL} < {[ {SiO}_{3}^{-} ]}_{B L} ; \\ {[ {SiO}_{3}^{-} ]}_{SL} < {[ {SiO}_{3}^{-} ]}_{B L} ; and / or \\ {[ {SiO}_{3}^{-} ]}_{G L} < {[ {SiO}_{3}^{-} ]}_{PL} ;\end{matrix}$$

wherein [SiO₃⁻]_{GL} are the counts of [SiO₃⁻] ions in the gliding layer, measured by a TOF-SIMS;
wherein [SiO₃⁻]_{PL} are the counts of [SiO₃⁻] ions in the protection layer, measured by a TOF-SIMS;
wherein [SiO₃⁻]_{BL} are the counts of [SiO₃⁻] ions in the barrier layer, measured by a TOF-SIMS;
wherein [SiO₃⁻]_{SL} are the counts of [SiO₃⁻] ions in the smoothing layer, measured by a TOF-SIMS.
   In one embodiment of the coated element, one or more of the following conditions is/are fulfilled:

$$\begin{matrix}i ) & {[ Si {C}^{-} ]}_{P L} > {[ Si {C}^{-} ]}_{B L} ; \\ ii ) & {[ Si {C}^{-} ]}_{P L} < {[ {SiC}^{-} ]}_{SL} ; or {[ Si {C}^{-} ]}_{P L} > {[ Si {C}^{-} ]}_{SL} ; \\ iii ) & {[ Si {C}^{-} ]}_{P L} > {[ Si {C}^{-} ]}_{GL} ; and / or \\ iv ) & {[ Si {C}^{-} ]}_{GL} > {[ Si {C}^{-} ]}_{B L} ;\end{matrix}$$

wherein [SiC⁻]_{GL} are the counts of [SiC⁻] ions in the gliding layer, measured by a TOF-SIMS;
wherein [SiC⁻]_{PL} are the counts of [SiC⁻] ions in the protection layer, measured by a TOF-SIMS;
wherein [SiC⁻]_{BL} are the counts of [SiC⁻] ions in the barrier layer, measured by a TOF-SIMS;
wherein [SiC⁻]_{SL} are the counts of [SiC⁻] ions in the smoothing layer, measured by a TOF-SIMS.

In one embodiment of the coated element, one or more of the following conditions is/are fulfilled:

$$\begin{matrix}{[ {Si {C}_{2} H}^{+} ]}_{B L} < {[ {Si {C}_{2} H}^{+} ]}_{S L} ; \\ {[ {Si {C}_{2} H}^{+} ]}_{GL} < {[ {Si {C}_{2} H}^{+} ]}_{S L} ; \\ {[ {Si {C}_{2} H}^{+} ]}_{B L} < {[ {Si {C}_{2} H}^{+} ]}_{PL} ; \\ {[ {Si {C}_{2} H}^{+} ]}_{GL} < {[ {Si {C}_{2} H}^{+} ]}_{PL} ; and / or \\ {[ {Si {C}_{2} H}^{+} ]}_{GL} > {[ {SiC}_{2} {H}^{+} ]}_{BL} ;\end{matrix}$$

wherein [SiC₂H⁺]_{GL} are the counts of [SiC₂H⁺] ions in the gliding layer, measured by a TOF-SIMS;
wherein [SiC₂H⁺]_{PL} are the counts of [SiC₂H⁺] ions in the protection layer, measured by a TOF-SIMS;
wherein [SiC₂H⁺]_{BL} are the counts of [SiC₂H⁺] ions in the barrier layer, measured by a TOF-SIMS;
wherein [SiC₂H⁺]_{SL} are the counts of [SiC₂H⁺] ions in the smoothing layer, measured by a TOF-SIMS.
   In one embodiment of the coated element, one or more of the following conditions is/are fulfilled:

$$\begin{matrix}{[ {SiO}^{+} ]}_{G L} < {[ Si {O}^{+} ]}_{B L} ; \\ {[ {SiO}^{+} ]}_{P L} < {[ Si {O}^{+} ]}_{B L} ; \\ {[ {SiO}^{+} ]}_{SL} < {[ Si {O}^{+} ]}_{B L} ; and / or \\ {[ {SiO}^{+} ]}_{G L} < {[ Si {O}^{+} ]}_{PL} ;\end{matrix}$$

wherein [SiO⁺]_{GL} are the counts of [SiO⁺] ions in the gliding layer, measured by a TOF-SIMS;
wherein [SiO⁺]_{PL} are the counts of [SiO⁺] ions in the protection layer, measured by a TOF-SIMS;
wherein [SiO⁺]_{BL} are the counts of [SiO⁺] ions in the barrier layer, measured by a TOF-SIMS;
wherein [SiO⁺]_{SL} are the counts of [SiO⁺] ions in the smoothing layer, measured by a TOF-SIMS.

In one embodiment of the coated element, the thickness of the coating is 1 nm to 1 mm, 10 nm to 0.5 mm, 200 nm to 2000 nm, or 500 nm to 1500 nm.

In one embodiment of the coated element, the smoothing layer, the barrier layer, the protection layer and/or the gliding layer, more preferably all layers, comprise(s), preferably consist(s) of, the elements Si, O and C measured by XPS.

In one embodiment of the coated element, the breaking force of the coated element is 1 N to 18 N, preferably 1.5 N to 10 N, or 2.0 N to 7 N. In one embodiment of the coated element, the breaking force of the coated element is 1 N or more, 1.5 N or more, or 2.0 N or more. In one embodiment of the coated element, the breaking force of the coated element is 18 N or less, 10 N or less, 7 N or less, or 5 N or less.

In one embodiment of the coated element, the gliding force of the coated element is 0.1 N to 10.0 N, preferably 0.3 N to 7.0 N, more preferably 0.4 N to 5.0 N, more preferably 0.5 N to 4.0 N, more preferably 1.0 N to 3.0 N. In one embodiment of the coated element, the gliding force of the coated element is 0.1 N or more, 0.3 N or more, 0.4 N or more, 0.5 N or more, or 1.0 N or more. In one embodiment of the coated element, the gliding force of the coated element is 10.0 N or less, 7.0 N or less, 5.0 N or less, 4.0 N or less, or 3.0 N or less.

In one embodiment of the coated element, one or more of the following equation(s) are fulfilled:

$$\begin{matrix}BF 7 / BF 1 \leq bf ; and / or \\ BF 28 / BF 1 \leq bf ; and / or \\ BF 28 / BF 7 \leq bf ;\end{matrix}$$

wherein BF1 is the breaking force after 1 day,
wherein BF7 is the breaking force after 7 days,
wherein BF28 is the breaking force after 28 days,
wherein bf is 4.0, preferably 3.0, more preferably 2.0, more preferably 1.8, more preferably 1.6, more preferably 1.4, more preferably 1.2.

In one embodiment of the coated element, the smoothing layer, the barrier layer, the protection layer and/or the gliding layer, more preferably all layers, is/are obtainable by the method according to any one of the following items.

### System or Kit

In one aspect a system or a kit is provided, the system or the kit comprising:
the coated element according to this disclosure, wherein the element is a syringe or cartridge, and
a plunger, preferably and a plunger rod;
preferably a closure, more preferably a tip cap and/or a needle shield.

### Closed System

In one aspect a closed system is provided, the closed system comprising:
the coated element according to this disclosure, wherein the element is a syringe or cartridge, and
a plunger, preferably a plunger rod;
preferably a closure, more preferably a tip cap and/or a needle shield; and wherein the closed coated element passes the container closure integrity test according to DIN EN ISO 8871-5:2016; chapter 4.4 in combination with Annex D.

In one embodiment, the closed system comprises a composition, preferably a pharmaceutical composition, more preferably a composition containing biologics or mRNA.

### Method

In one aspect a method for coating an element is provided, preferably a method to obtain a coated element according to this disclosure, comprising the steps:
providing an element comprising a surface;
applying one or more layers, preferably a smoothing layer, a barrier layer, a protection layer and optionally a gliding layer; by performing a coating process on at least part of the surface, comprising the steps:
   a) surrounding the at least part of the surface of the element with a precursor; and
   b) irradiating the precursor to generate a plasma.

In one embodiment of the method, the coating process is a plasma-enhanced chemical vapor deposition (PECVD) process, plasma impulse chemical vapor deposition (PICVD) process or plasma assisted chemical vapor deposition (PACVD) process, preferably a plasma impulse chemical vapor deposition (PICVD) process.

In a most preferred embodiment of the method, the coating process is a plasma impulse chemical vapor deposition (PICVD) process.

In one embodiment of the method, the precursor comprises one or more of hexamethyldisiloxane (HMDSO), hexamethyldisilazane (HMDSN), tetramethylsilane (TMS), trimethylborazole (TMB), tri(dimethylaminosilyl)-amino-di(dimethylamino)borane (TDADB), tris(trimethylsilyl)borate (TMSB), hexamethylcyclotrisiloxan (HMCTSO), octamethylcyclotetrasiloxan (OMCTS), decamethylcyclopentasiloxan (DMCPS), dodecamethylcyclohexasiloxan (DMCHS) diacetoxy-di-t-butoxysilane (DADBS), tetraethoxysilane (TEOS), tris(trimethylsilyloxy)vinylsilane (TTMSVS), vinyltriethoxysilane (VTES) and/or combinations thereof, preferably the precursor is HMDSO.

In one embodiment of the method, the precursor comprises, preferably consists of, the elements Si, C, O and H.

In one embodiment of the method, for the application of the smoothing layer, the barrier layer, the protection layer and/or the gliding layer, one or more of the following conditions is/are fulfilled:
i. wherein the coating process is a plasma impulse chemical vapor deposition (PICVD) process,
ii. wherein the irradiation is carried out by a microwave generator, preferably wherein the ray has a frequency of 300 MHz to 300 GHz, more preferably 600 MHz to 100 GHz, more preferably 800 MHz to 10 GHz, more preferably 900 MHz to 3 GHz, more preferably 2.45 GHz; and/or
iii. wherein the input power IP, preferably the input power IP of the microwave generator, is 100 W to 10000 W, preferably 300 W to 9000 W, more preferably 500 W to 8000 W, more preferably 700 W to 6000 W, more preferably 900 W to 4000 W, more preferably 1100 W to 2000 W; and/or
iv. wherein the precursor P comprises one or more of hexamethyldisiloxane (HMDSO), hexamethyldisilazane (HMDS), tetramethylsilane (TMS), trimethylborazole (TMB), tri(dime-thylaminosilyl)-amino-di(dimethylamino)borane (TDADB), tris(trimethylsilyl)borate (TMSB), hexamethylcyclotrisiloxan (HMCTSO), octamethylcyclotetrasiloxan (OMCTS), decamethylcyclopentasiloxan (DMCPS), dodecamethylcyclohexasiloxan (DMCHS) diacetoxy-di-t-butoxysilane (DADBS), tetraethoxysilane (TEOS), tris(trimethylsilyloxy)vinylsilane (TTMSVS), vinyltriethoxysilane (VTES) and/or combinations thereof, preferably the precursor P is HMDSO; and/or
   wherein, besides the precursor P, a gas, preferably oxygen, is fed.

It has since long been accepted that the frequency at which a high frequency dis-charge is sustained has considerable influence on the properties of the plasma (Moisan, M., Barbeau, C., Claude, R., Ferreira, C. M., Margot, J., Paraszczak, J., Sá, A. B., Sauvé, G., & Wertheimer, M. R. (1991). Radio frequency or microwave plasma reactors? Factors determining the optimum frequency of operation. Journal of Vacuum Science & Technology B: Microelectronics and Nanometer Structures Processing, Measurement, and Phenomena 9(1), 8-25). The generated electron density for a given high-frequency power density deposited into the plasma is higher at microwave frequencies than at radio frequencies. Based on their study, the authors have established that converting a plasma process from 13.56 to 2450 MHz does not necessarily lead to the greatest possible process enhancement, and that optimization may require the plasma reactor to be constructed in such a way as to allow the excitation frequency to be changed.

The latter task of (re) constructing a plasma reactor to allow a change of the excitation frequency is by no means trivial and has to the knowledge of the applicant not been implemented to date and not been shared as public knowledge.

Pulsed microwave plasma with low pulsing frequencies, e.g. of about 100 Hz and a low duty cycle (D≤0.1), as employed in the present disclosure, are advantageous for optical and other functional coatings. In the PICVD process, dielectric substrates may be typically placed directly on the microwave window, while at a relatively high pressure on the order of 1 torr or less, a very dense plasma is formed near the substrate during a very short pulse of typically 1 to 100 ms in duration.

Radjef et al. (Characterising a Custom-Built Radio Frequency PECVD Reactor to Vary the Mechanical Properties of TMDSO Films. Molecules 26, 2021, 5621) investigated radio-frequency (RF) PECVD in comparison to microwave (MW) PECVD for the deposition of organosiloxane films based on TMDSO as a precursor, and found that the majority of the MW TMDSO films were slightly smoother than the RF TMDSO films, with R_{q} values as low as 0.4 nm. Generally, a higher concentration of O₂ within the system resulted in greater fragmentation of the TMDSO molecule. It was speculated that a higher concentration of O₂ results in the efficient cleavage of the methyl groups, leaving a relatively bare O—Si—O backbone, and that such fragmentation results in a stiffer, more ‘silica-like’ film with a crosslinked network of O—Si—O.

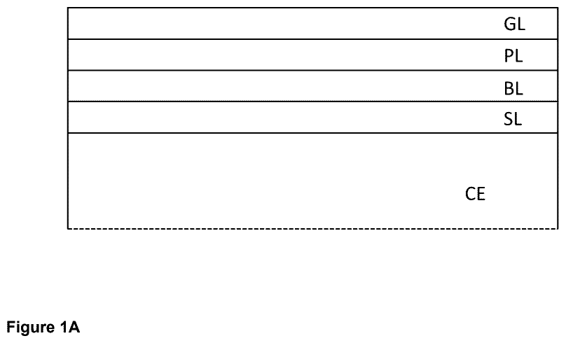

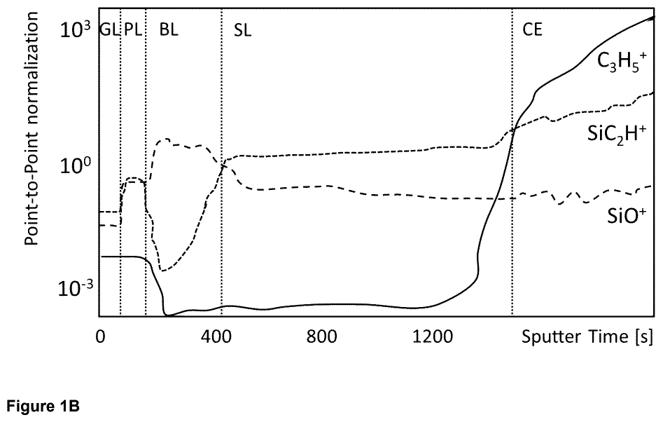

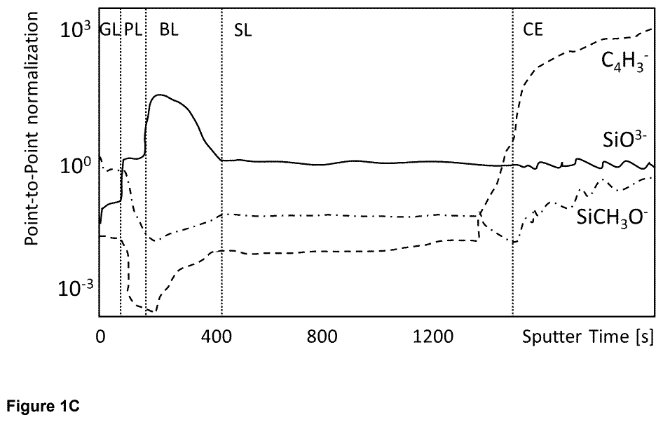

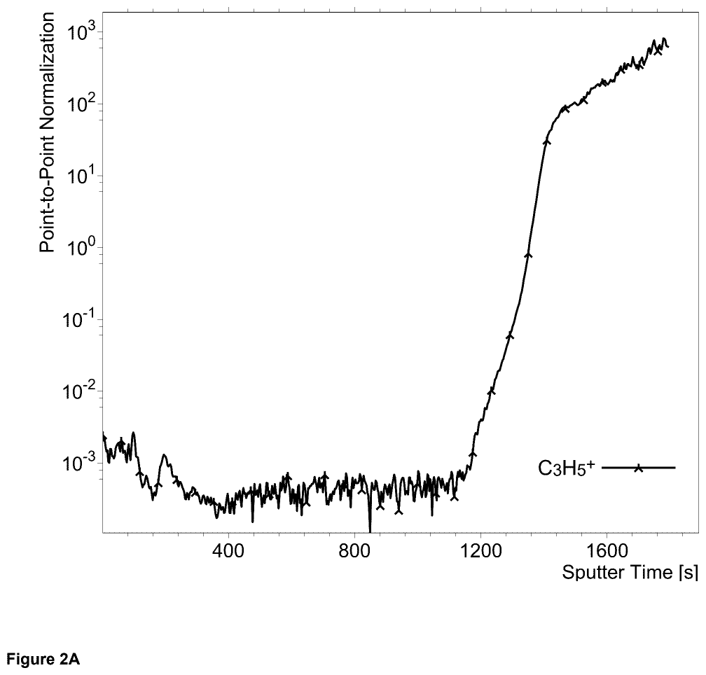

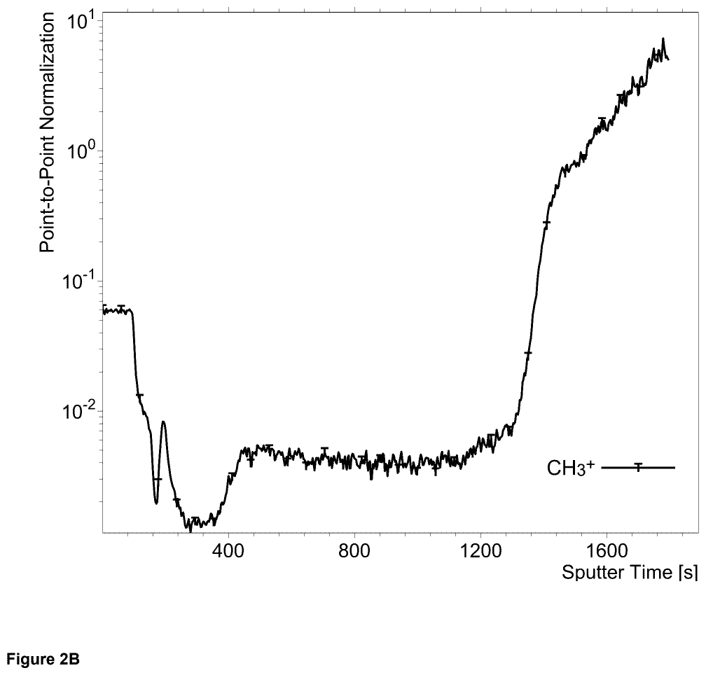

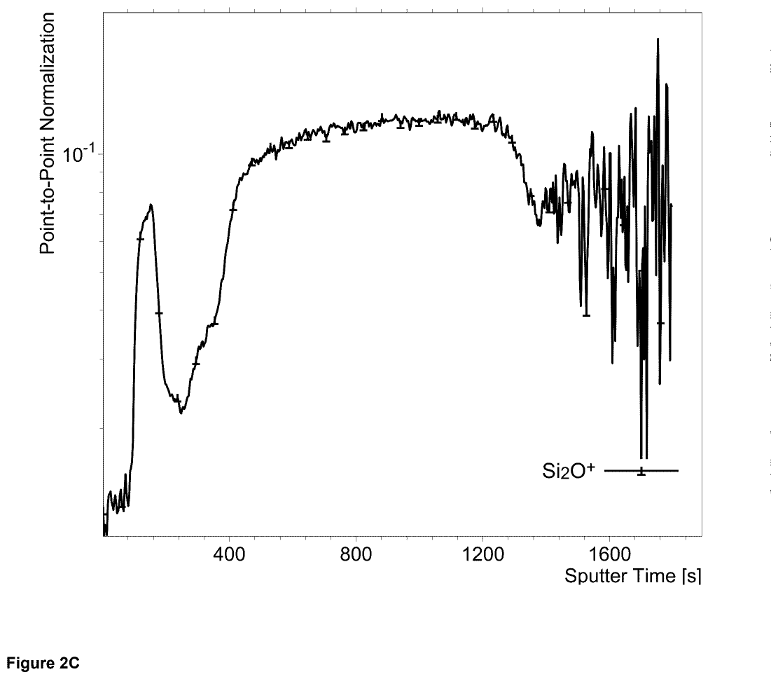

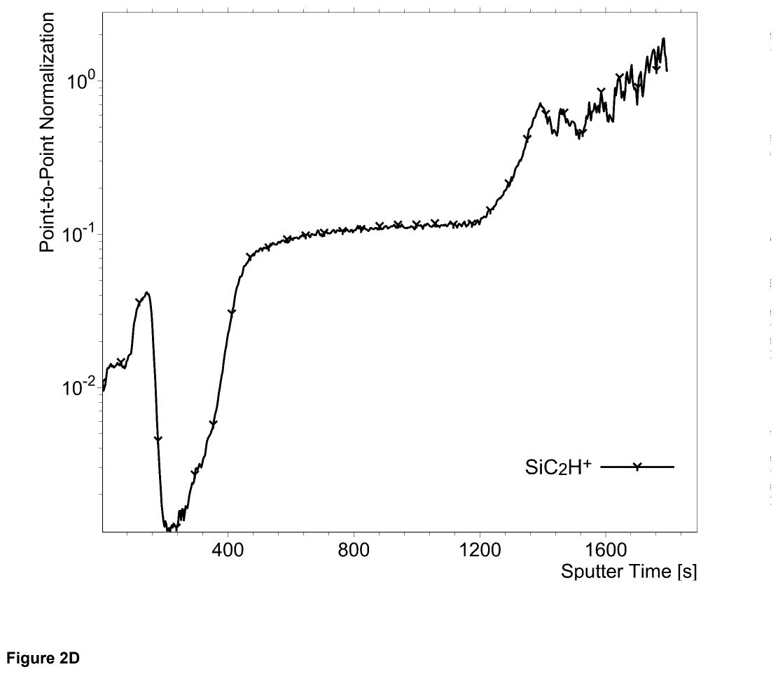

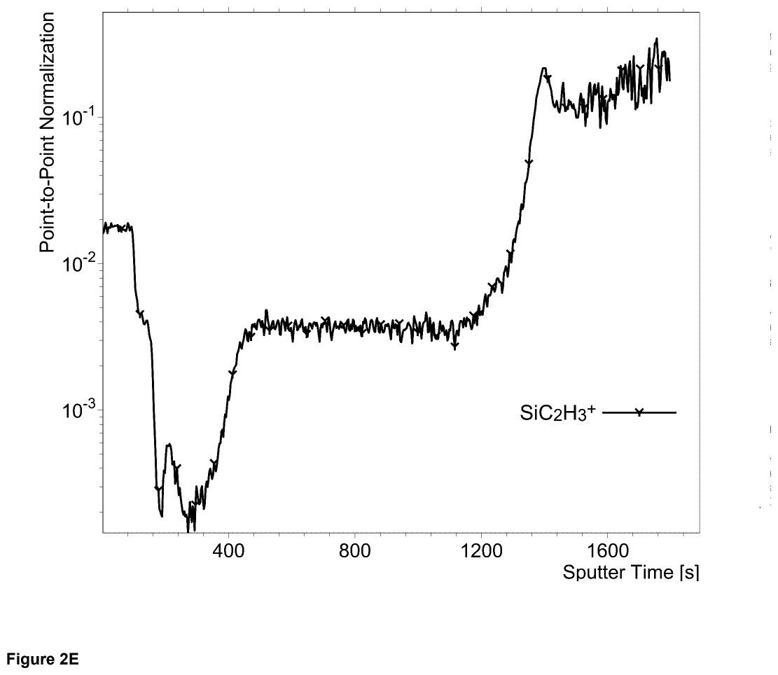

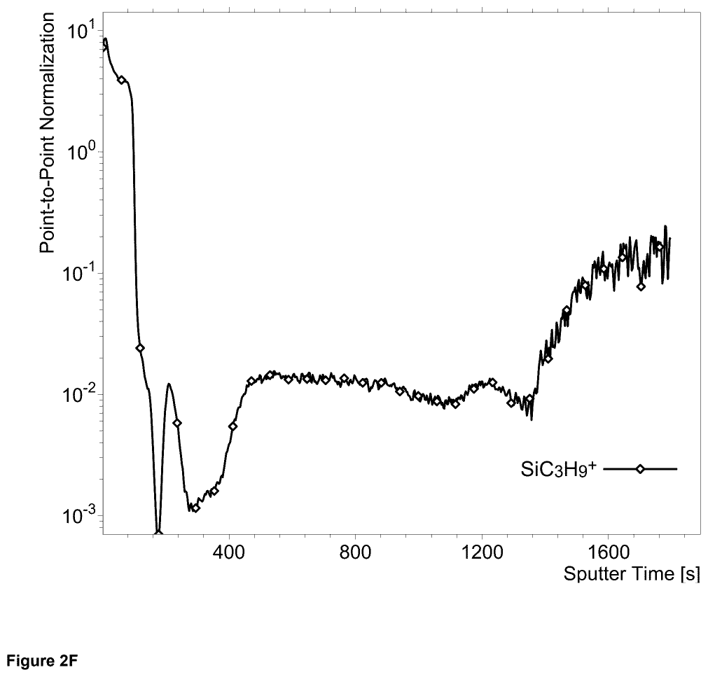

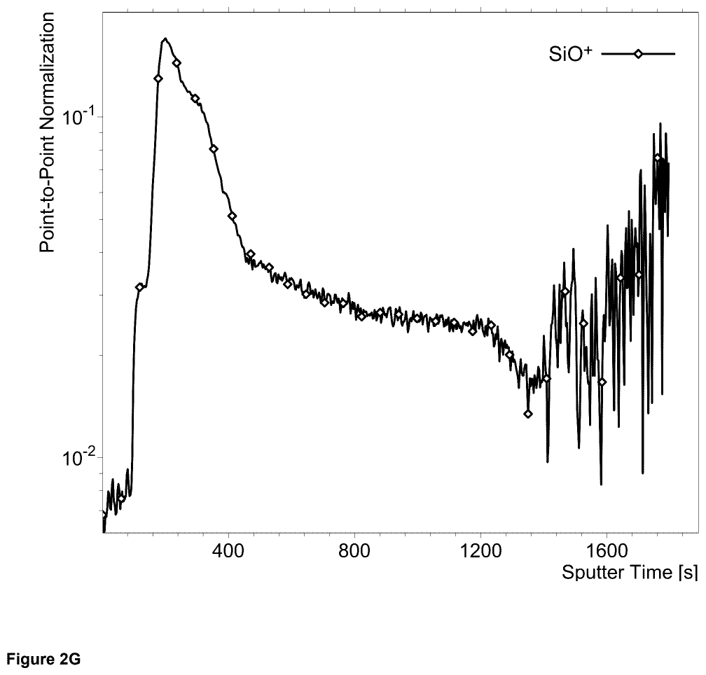

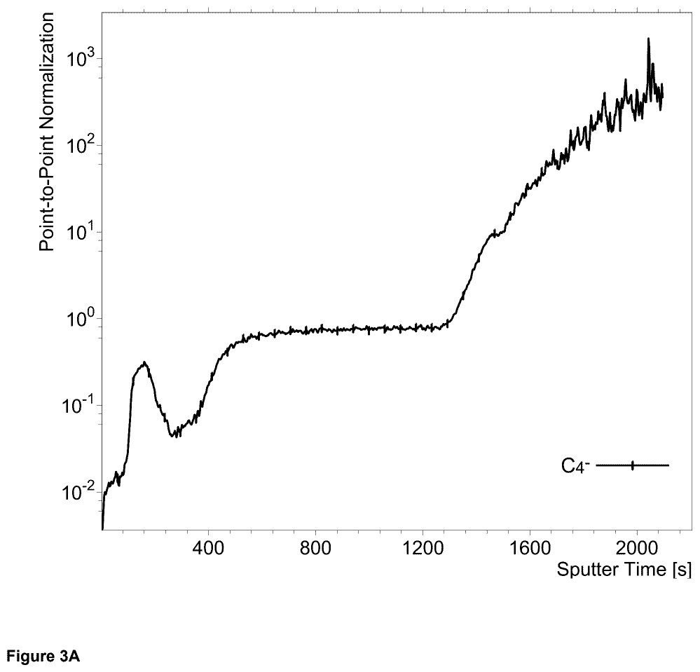

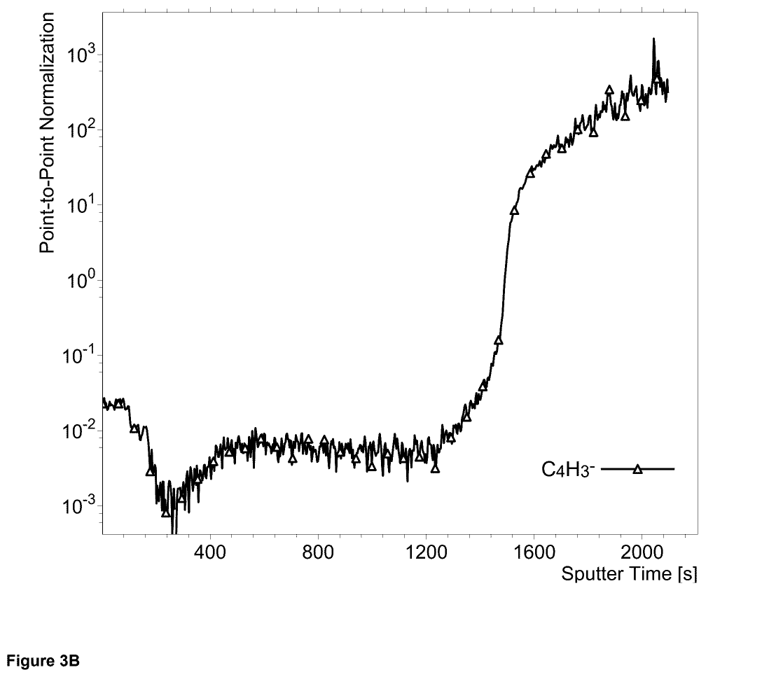

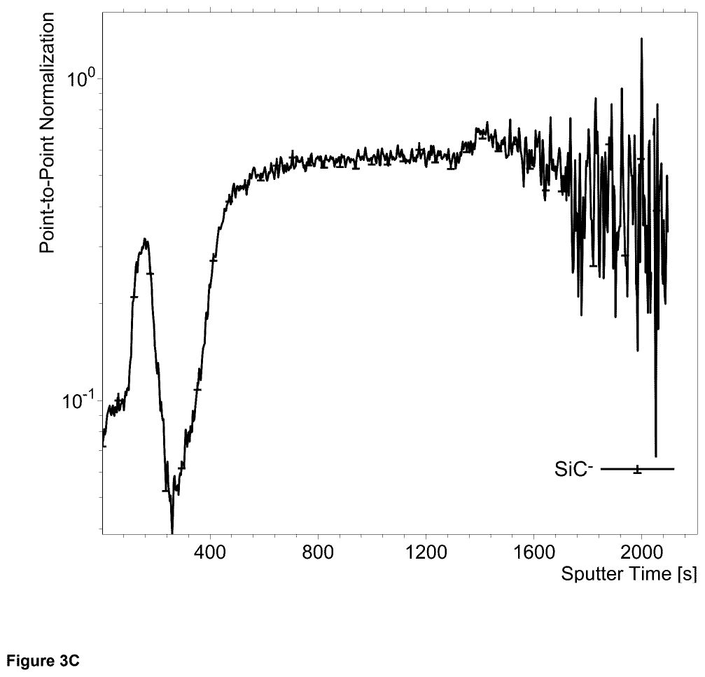

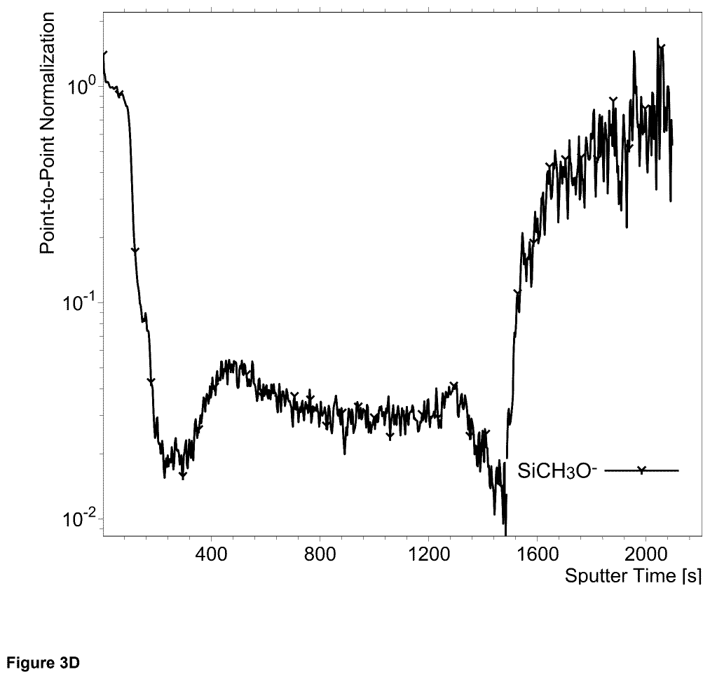

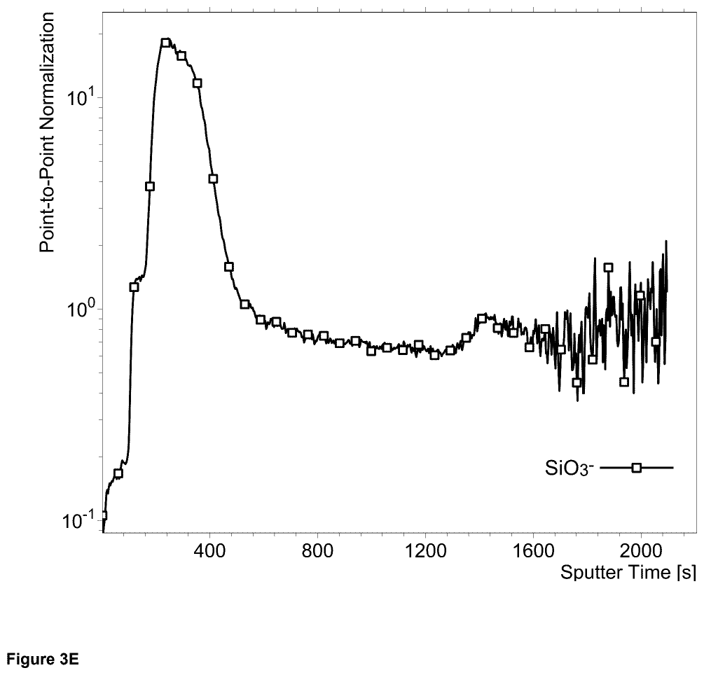

The inventors of the present disclosure have established a process and process conditions which leaves intact the chosen precursor to a greater extent. The layer structure of the coated element includes, in this order, a smoothing layer (SL), a barrier layer (BL), a protection layer (PL), and an optional gliding layer (GL). The coated element may be a polymer substrate. Each of the said layers is chemically distinct and is characterised by the detection of specific ToF-SIMS species, i.e. SiC⁻, C₄⁻C₄H₃⁻, SiCH₃O⁻; and SiO₃⁻ in negative mode, and C₃H₅⁺, SiO⁺. SiC₂H⁺, SiC₂H₃⁺, Si₂O⁺, SiC₃H₉⁺, and CH₃⁺ in positive mode. The exact qualitative and quantitative characteristics depend on both the PICVD parameters employed during each of the four deposition steps (1 to 4) in addition to the chosen precursor, and the sputter and analysis parameters of the ToF-SIMS measurement (cf. FIG. 1B and FIG. 1C).

Without wishing to be bound to theory, the ratio [ms/ms] of all pulse durations PD_{LAYER} [ms] to all pulse pauses PP_{LAYER} is an important parameter to control and adjust the deposition of each of the three or four individual layers, as part of the PICVD process. This parameter may be alternative expressed as the ‘duty cycle’ or ‘power cycle’ which is the fraction of one period in which a signal or system is active. The duty cycle may be expressed as a percent-age or ratio. A period is defined as the time it takes for a signal to complete an on-and-off cycle.

In one advantageous embodiment of the method, the application of the smoothing layer is performed at a ratio [ms/ms] of all pulse durations PD_{SL} [ms] to all pulse pauses PP_{SL} [ms] of 0.01 to 0.5, the application of the barrier layer is performed at a ratio [ms/ms] of all pulse durations PD_{BL} [ms] to all pulse pauses PP_{BL} [ms] is 0.01 to 1, preferably 0.1 to 0.8, and/or the application of the protection layer is performed at a ratio [ms/ms] of all pulse durations PD_{PL} [ms] to all pulse pauses PP_{PL} [ms] is 0.001 to 1, preferably 0.01 to 0.5.

In one embodiment of the method, for the application of the smoothing layer one or more of the following conditions is/are fulfilled:
i. wherein the process temperature PT_{SL} is 15° C. to 150° C., 20° C. to 80° C., or 25° C. to 40° C.; and/or
ii. wherein the pulse duration PD_{SL} of the plasma is 0.0001 ms to 1.5 ms, 0.1 ms to 1.2 ms, or preferably 0.3 ms to 0.8 ms; and/or
iii. the pulse pause PP_{SL} between two pulses is 0.01 ms to 100 ms, 0.1 ms to 70 ms, 1.0 ms to 50 ms, or 5.0 ms to 20 ms; and/or
iv. the total time TT_{SL} of irradiation is 1 s to 50 s, 2 s to 20 s, 3 s to 10 s, or 4 s to 7 s; and/or
v. the ratio [ms/ms] of all pulse durations PD_{SL} [ms] to all pulse pauses PP_{SL} [ms] is 0.001 to 1, 0.01 to 0.5, or 0.03 to 0.08; and/or
vi. wherein the process pressure PR_{SL} is 0.1 mbar to 100 mbar, 0.3 mbar to 10 mbar, 0.5 to 3.0 mbar, or 0.5 to 0.8 mbar; and/or
vii. wherein the flow rate of the precursor P is 1 sccm to 200 sccm, 5 sccm to 100 sccm, or 15 sccm to 30 sccm,
   wherein, besides the precursor, no further substance (such as a gas) is fed.

In one embodiment of the method, for the application of the barrier layer one or more of the following conditions is/are fulfilled:
i. wherein the process temperature PT_{BL} is 15° C. to 200° C., or 25° C. to 70° C.; and/or
ii. wherein the pulse duration PD_{BL} of the plasma is 0.5 ms to 50 ms, 1.0 to 10 ms, or 1.5 ms to 2.5 ms; and/or
iii. the pulse pause PP_{BL} between two pulses is 0.05 ms to 500 ms, 0.1 ms to 100 ms, 1.0 ms to 30 ms, or 3.0 ms to 10 ms; and/or
iv. the total time TT_{BL} of irradiation is 1 s to 50 s, 3 s to 30 s, 5 s to 15 s, or 6 s to 12 s; and/or
v. the ratio [ms/ms] of all pulse durations PD_{BL} [ms] to all pulse pauses PP_{BL} [ms] is 0.01 to 1, preferably 0.1 to 0.8, more preferably 0.3 to 0.6; and/or
vi. wherein the process pressure PR_{BL} is 0.01 mbar to 10 mbar, 0.1 mbar to 5 mbar, 0.2 mbar to 3.0 mbar, or 0.25 mbar to 0.8 mbar; and/or
vii. wherein the flow rate of the precursor P is 0.01 sccm to 20 sccm, 0.1 sccm to 5.0 sccm, or 0.3 sccm to 0.9 sccm,
   wherein, besides the precursor, oxygen gas is fed and the flow rate of the oxygen gas is 0.1 to 100 sccm, 5 sccm to 80 sccm, or 30 sccm to 50 sccm.

In one embodiment of the method, for the application of the protection layer one or more of the following conditions is/are fulfilled:
i. wherein the process temperature PT_{PL} is 15° C. to 150° C., 30° C. to 100° C., or 40° C. to 70° C.; and/or
ii. wherein the pulse duration PD_{PL} of the plasma is 0.001 ms to 1.5 ms, 0.01 ms to 1.0 ms, or 0.3 ms to 0.8 ms; and/or
iii. the pulse pause PP_{PL} between two pulses is 0.01 ms to 100 ms, 1 ms to 50 ms, 3 ms to 20 ms, 5 ms to 15 ms, or 8 ms to 12 ms; and/or
iv. the total time TT_{PL} of irradiation is 0.1 s to 20 s, 0.3 s to 10 s, 0.7 s to 8 s, or 0.8 s to 1.2 s; and/or
v. the ratio [ms/ms] of all pulse durations PD_{PL} [ms] to all pulse pauses PP_{PL} [ms] is 0.001 to 1, preferably 0.01 to 0.5, more preferably 0.03 to 0.08; and/or
vi. wherein the process pressure PR_{PL} is 0.1 mbar to 100 mbar, 0.1 mbar to 20 mbar, 0.3 mbar to 1.0 mbar, or 0.5 mbar to 0.8 mbar; and/or
vii. wherein the flow rate of the precursor PPL is 1 sccm to 200 sccm, 5 sccm to 40 sccm, or 10 sccm to 20 sccm,
   wherein, besides the precursor, oxygen gas is fed and the flow rate of the gas is 0.1 to 100, preferably 2 sccm to 30 sccm, more preferably 4 sccm to 8 sccm.

In one embodiment of the method, for the application of the gliding layer one or more of the following conditions is/are fulfilled:
i. wherein the process temperature PT_{GL} is 15° C. to 150° C., 25° C. to 100° C., or 40° C. to 70° C.; and/or
ii. wherein the pulse duration PD_{GL} of the plasma is 0.001 ms to 1.5 ms, 0.01 ms to 0.8 ms, or 0.02 ms to 0.1 ms; and/or
iii. the pulse pause PP_{GL} between two pulses is 0.001 ms to 100 ms, 0.01 ms to 20 ms, 0.1 ms to 10 ms, 0.3 ms to 3 ms; and/or
iv. the total time TT_{GL} of irradiation is 1 s to 50 s, 3 s to 30 s, 5 s to 15 s, or 6 s to 10 s; and/or
v. the ratio [ms/ms] of all pulse durations PD_{GL} [ms] to all pulse pauses PP_{GL} [ms] is 0.001 to 1, preferably 0.01 to 0.5, more preferably 0.02 to 0.08; and/or
vi. wherein the process pressure PR_{GL} is 0.1 mbar to 100 mbar, 0.2 mbar to 10 mbar, 0.3 to 1.5 mbar, or 0.6 to 1.0 mbar; and/or
vii. wherein the flow rate of the precursor PGL is 1 sccm to 200 sccm, 3 sccm to 50 sccm, or 5 sccm to 15 sccm,
   wherein, besides the precursor, no further substance is fed.

### Method (Further Embodiments)

In one aspect a method for coating an element is provided, preferably a method to obtain a coated element according to this disclosure, comprising the steps:
providing an element comprising a surface;
applying a smoothing layer, a barrier layer, a protection layer and optionally a gliding layer; by performing a coating process on at least part of the surface, comprising the steps:
   a) surrounding the at least part of the surface of the element with a precursor; and
   b) irradiating the precursor to generate a plasma,
   wherein the following conditions are fulfilled:
i. the coating process is a plasma impulse chemical vapor deposition (PICVD) process,
ii. the irradiation is carried out by a microwave generator, wherein the ray has a frequency of MHz to 3 GHz, more preferably 2.45 GHz; and/or
iii. the input power IP of the microwave generator is 900 W to 2000 W; and/or
iv. wherein the precursor P comprises one or more of hexamethyldisiloxane (HMDSO), hexamethyldisilazane (HMDS), tetramethylsilane (TMS), trimethylborazole (TMB), tri(dime-thylaminosilyl)-amino-di(dimethylamino)borane (TDADB), tris(trimethylsilyl)borate (TMSB), hexamethylcyclotrisiloxan (HMCTSO), octamethylcyclotetrasiloxan (OMCTS), decamethylcyclopentasiloxan (DMCPS), dodecamethylcyclohexasiloxan (DMCHS) diacetoxy-di-t-butoxysilane (DADBS), tetraethoxysilane (TEOS), tris(trimethylsilyloxy)vinylsilane (TTMSVS), vinyltriethoxysilane (VTES) and/or combinations thereof, preferably the precursor P is HMDSO,
   wherein, besides the precursor P, oxygen gas is fed during the application of the barrier layer.

In one embodiment of the method, for the application of the smoothing layer the following conditions are fulfilled:
i. the process temperature PT_{SL} is 15° C. to 150° C.;
ii. the pulse duration PD_{SL} of the plasma is 0.0001 ms to 1.5 ms;
iii. the pulse pause PP_{SL} between two pulses is 0.01 ms to 100 ms;
iv. the total time TT_{SL} of irradiation is 1 s to 50 s;
v. the ratio [ms/ms] of all pulse durations PD_{SL} [ms] to all pulse pauses PP_{SL} [ms] is 0.001 to 1;
vi. the process pressure PR_{SL} is 0.1 mbar to 100 mbar;
vii. the flow rate of the precursor P is 1 sccm to 200 sccm,
   wherein, besides the precursor, no further substance (such as a gas) is fed.

In one embodiment of the method, for the application of the smoothing layer the following conditions are fulfilled:
i. the process temperature PT_{SL} is 25° C. to 40° C.;
ii. the pulse duration PD_{SL} of the plasma is 0.3 ms to 0.8 ms;
iii. the pulse pause PP_{SL} between two pulses is 5.0 ms to 20 ms;
iv. the total time TT_{SL} of irradiation is 4 s to 7 s;
v. the ratio [ms/ms] of all pulse durations PD_{SL} [ms] to all pulse pauses PP_{SL} [ms] is 0.03 to 0.08;
vi. the process pressure PR_{SL} is 0.5 to 0.8 mbar;
vii. the flow rate of the precursor P is 15 sccm to 30 sccm,
   wherein, besides the precursor, no further substance (such as a gas) is fed.

In one embodiment of the method, for the application of the barrier layer the following conditions are fulfilled:
i. the process temperature PT_{BL} is 15° C. to 200° C.;
ii. the pulse duration PD_{BL} of the plasma is 0.5 ms to 50 ms;
iii. the pulse pause PP_{BL} between two pulses is 0.05 ms to 500 ms;
iv. the total time TT_{BL} of irradiation is 1 s to 50 s;
v. the ratio [ms/ms] of all pulse durations PD_{BL} [ms] to all pulse pauses PP_{BL} [ms] is 0.01 to 1;
vi. the process pressure PR_{BL} is 0.01 mbar to 10 mbar;
vii. the flow rate of the precursor P is 0.01 sccm to 20 sccm,
   wherein, besides the precursor, oxygen gas is fed and the flow rate of the oxygen gas is 0.1 to 100 sccm.

In one embodiment of the method, for the application of the barrier layer the following conditions are fulfilled:
i. the process temperature PT_{BL} is 25° C. to 70° C.;
ii. the pulse duration PD_{BL} of the plasma is 1.5 ms to 2.5 ms;
iii. the pulse pause PP_{BL} between two pulses is 3.0 ms to 10 ms;
iv. the total time TT_{BL} of irradiation is 6 s to 12 s;
v. the ratio [ms/ms] of all pulse durations PD_{BL} [ms] to all pulse pauses PP_{BL} [ms] is 0.3 to 0.6;
vi. the process pressure PR_{BL} is 0.25 mbar to 0.8 mbar;
vii. the flow rate of the precursor P is 0.3 sccm to 0.9 sccm,
   wherein, besides the precursor, oxygen gas is fed and the flow rate of the oxygen gas is 30 sccm to 50 sccm.

In one embodiment of the method, for the application of the protection layer the following conditions are fulfilled:
i. the process temperature PT_{PL} is 15° C. to 150° C.;
ii. the pulse duration PD_{PL} of the plasma is 0.001 ms to 1.5 ms;
iii. the pulse pause PP_{PL} between two pulses is 0.01 ms to 100 ms;
iv. the total time TT_{PL} of irradiation is 0.1 s to 20 s;
v. the ratio [ms/ms] of all pulse durations PD_{PL} [ms] to all pulse pauses PP_{PL} [ms] is 0.001 to 1;
vi. the process pressure PR_{PL} is 0.1 mbar to 100 mbar; and
vii. the flow rate of the precursor PPL is 1 sccm to 200 sccm,
   wherein, besides the precursor, oxygen gas is fed and the flow rate of the gas is 0.1 to 100 sccm.

In one embodiment of the method, for the application of the protection layer the following conditions are fulfilled:
i. the process temperature PT_{PL} is 40° C. to 70° C.;
ii. the pulse duration PD_{PL} of the plasma is 0.3 ms to 0.8 ms;
iii. the pulse pause PP_{PL} between two pulses is 8 ms to 12 ms;
iv. the total time TT_{PL} of irradiation is 0.8 s to 1.2 s;
v. the ratio [ms/ms] of all pulse durations PD_{PL} [ms] to all pulse pauses PP_{PL} [ms] is 0.03 to 0.08;
vi. the process pressure PR_{PL} is 0.5 mbar to 0.8 mbar; and
vii. the flow rate of the precursor PPL is 10 sccm to 20 sccm,
   wherein, besides the precursor, oxygen gas is fed and the flow rate of the gas is 4 sccm to 8 sccm.

In one embodiment of the method, for the application of the gliding layer the following conditions are fulfilled:
i. the process temperature PT_{GL} is 15° C. to 150° C.;
ii. the pulse duration PD_{GL} of the plasma is 0.001 ms to 1.5 ms;
iii. the pulse pause PP_{GL} between two pulses is 0.001 ms to 100 ms;
iv. the total time TT_{GL} of irradiation is 1 s to 50 s;
v. the ratio [ms/ms] of all pulse durations PD_{GL} [ms] to all pulse pauses PP_{GL} [ms] is 0.001 to 1;
vi. the process pressure PR_{GL} is 0.1 mbar to 100 mbar; and
vii. the flow rate of the precursor PGL is 1 sccm to 200 sccm,
   wherein, besides the precursor, no further substance is fed.

In one embodiment of the method, for the application of the gliding layer the following conditions are fulfilled:
i. the process temperature PT_{GL} is 40° C. to 70° C.;
ii. the pulse duration PD_{GL} of the plasma is 0.02 ms to 0.1 ms;
iii. the pulse pause PP_{GL} between two pulses is 0.3 ms to 3 ms;
iv. the total time TT_{GL} of irradiation is 6 s to 10 s;
v. the ratio [ms/ms] of all pulse durations PD_{GL} [ms] to all pulse pauses PP_{GL} [ms] is 0.02 to 0.08;
vi. the process pressure PR_{GL} is 0.6 to 1.0 mbar; and
vii. the flow rate of the precursor PGL is 5 sccm to 15 sccm,
   wherein, besides the precursor, no further substance is fed.

### Pharmaceutical Container

In one embodiment of the coated element according to this disclosure or the method according to this disclosure, the element is a pharmaceutical container, preferably a vial, ampule, cartridge or syringe, more preferably a cartridge or syringe, most preferably a syringe.

In one embodiment, the element defines a lumen, wherein the brimful volume of the element is 0.1 ml to 200 ml, 0.2 ml to 100 ml, 0.4 ml to 50 ml, 0.6 ml to 20 ml, 0.8 ml to 15 ml, 1.0 ml to 10 ml, or 1.2 ml to 5 ml.

In one embodiment, the element comprises, preferably consists of glass or polymer, preferably polymer. In one embodiment, the element comprises or consists of cyclic olefin copolymer (COC) and/or cyclic olefin polymer (COP) and/or polypropylene (PP). In one embodiment, the element comprises or consists of cyclic olefin copolymer (COC).

### Silicone Coating

In one aspect, a method for coating an element is provided, preferably to obtain a coated element according to this disclosure, comprising the steps:
providing an element comprising a surface;
applying one or more layers, preferably the smoothing layer and the barrier layer, by performing a first coating process on at least part of the surface of the element to obtain a pre-coated element,
performing a second coating process comprising the steps:
   x) providing a silicone-forming composition, wherein the silicone-forming composition comprises:
i) a silicone compound suitable to form a silicone network after reaction;
ii) a catalyst suitable to catalyze a network reaction of the silicone compound;
iii) an inert silicone oil, wherein the inert silicone oil does not take part in the network reaction; and
iv) a diluent,
   wherein the diluent comprises a silicon-containing compound, wherein the silicone-forming composition comprises 45 to 95 wt. % diluent, and
   y) applying the silicone-forming composition to the pre-coated element, and reacting the silicone-forming composition
obtaining a coated element.

In one embodiment of the method, the second coating process, i.e. step y) is performed by curing the composition, preferably curing the composition by annealing to 50° C. to 300° C., or 80° C. to 120° C. and/or by applying IR irradiation.

In one embodiment of the method, i.e. the second coating process, i.e. step x), the diluent comprises a silicon-containing organic compound with 6 silicon atoms or less.

In one embodiment of the method, i.e. the second coating process, i.e. step x), the diluent has a surface tension of 19 mN/m or less.

In one embodiment of the method, i.e. the second coating process, i.e. step x), the diluent is chosen from the list of:
cyclic silicones, such as octamethylcyclotetrasiloxane, decamethylcyclopentasiloxane, dodecamethylcyclohexasiloxane, tetramethylcyclotetrasiloxane, pentamethylcyclopentasiloxane,
hexamethyldisiloxane (HMDSO),
octamethyltrisiloxane,
decamethyltetrasiloxane.

In one embodiment of the method, i.e. the second coating process, i.e. step x), the silicone-forming composition has a viscosity between 0.5 to 200 m·Pas, 1 to 50 m·Pas, or 1 to 10 m·Pas, at a temperature of 23° C. The viscosity can be measured using a rotational viscometer, e.g. described in DIN ISO 7884-2:1998-2.

In one embodiment of the method, i.e. the second coating process, i.e. step x), the silicone-forming composition has a contact angle of 28° or less, 24° or less, 18° or less, or 5° or less, 1 s after spreading the composition on a borosilicate glass surface at a temperature of 23° C., wherein the contact angle has been determined by drop shape analysis.

### Coated Container

In one aspect according to this disclosure, a coated container is provided,
wherein the coated container comprises an inner surface and an outer surface,
wherein at least a part of the inner surface is coated by a coating; and
wherein the coated container satisfies the following equation:

$$i / o \leq d$$

a value d ([mg/cm²]/[mg/cm²]) of 0.90, or 0.80, or 0.70, or 0.60, or 0.50, or 0.40, or 0.30, or 0.20, or 0.14, or 0.10, or 0.07,
wherein i is the leaching (mg/cm²) of one or more type(s) of ions and/or compounds obtainable by the method the method Lᵢ as described herein; and
wherein o is the leaching (mg/cm²) of one or more type(s) of ions and/or compounds of the base material of the coated container and/or is obtainable by the method Lₒ as described herein.

In one embodiment of the coated container, the one or more type(s) of ions and/or compounds are:
one or more compound(s) comprising C, preferably one or more compound(s) comprising C except compounds comprising Si; and/or
one or more monomer(s), preferably norbornene, norbornane, and/or bicyclopentane; and/or
one or more antioxidant(s), preferably wherein the antioxidant(s) is/are selected from the group consisting of phenolic antioxidants, more preferably pentaerythritol tetrakis(3-(3,5-di-tert-butyl-4-hydroxyphenyl)propionate and/or dibutylhydroxytoluene (BHT); phosphite antioxidants, more preferably tris(2,4-ditert-butylphenyl)phosphite; phosphonite antioxidants, preferably tetrakis(2,4-di-tert-butylphenyl)[1,1-biphenyl]-4,4′-diylbisphosphonite; and thioether antioxidants, more preferably wherein the antioxidant(s) is/are phenolic antioxidant(s), more preferably pentaerythritol tetrakis(3-(3,5-di-tert-butyl-4-hydroxyphenyl)propionate.

In one embodiment, the coated container or the coated element comprise a base material, wherein the base material comprises
one or more compound(s) comprising C, preferably one or more compound(s) comprising C except compounds comprising Si; and/or
one or more monomer(s), preferably norbornene, norbornane, and/or bicyclopentane; and/or
one or more antioxidant(s), preferably wherein the antioxidant(s) is/are selected from the group consisting of phenolic antioxidants, more preferably pentaerythritol tetrakis(3-(3,5-di-tert-butyl-4-hydroxyphenyl)propionate and/or dibutylhydroxytoluene (BHT); phosphite antioxidants, more preferably tris(2,4-ditert-butylphenyl)phosphite; phosphonite antioxidants, preferably tetrakis(2,4-di-tert-butylphenyl)[1,1-biphenyl]-4,4′-diylbisphosphonite; and thioether antioxidants, more preferably wherein the antioxidant(s) is/are phenolic antioxidant(s), more preferably pentaerythritol tetrakis(3-(3,5-di-tert-butyl-4-hydroxyphenyl)propionate.

### Method Lᵢ

In one embodiment of this disclosure, the following method (method Lᵢ) is provided, comprising the steps:
providing a container, preferably a coated container;
in case the container exhibits two openings, e.g. a syringe: covering the smaller opening with a closure, e.g. a tip cap;
filling 0.9× (brimful volume) of a 0.005 mol/l KOH-solution (KOH: Potassium hydroxide hydrate ≥99.995%, Suprapur® (Merck), demineralized water (ultrapure water of purity 1 analogue DIN ISO 3696 with ≤0.1 μS/cm at 25° C.)) in the pharmaceutical container;

closing the container with a closure seal, e.g. aluminum foil or a stopper;
inserting the container in an autoclave (e.g. Systec, model DX-150 (PM-CA-0001-01));
treating the container for 3 hours at 121° C. and 1 bar above ambient pressure, e.g. 2 bar;
opening the closure seal;
emptying the container;
filling and rinsing the container two times with deionized water;
in case the container is a glass container: filling 0.9× (brimful volume) of 0.1 mol/l HCl-solution (hydrochloric acid 30% Suprapur® (Merck)) diluted with demineralized water (ultrapure water of purity 1 analogue DIN ISO 3696 with ≤0.1 μS/cm at 25° C.)), i.e. the same volume as used in the alkaline treatment, in the pharmaceutical container, or
in case the container is a polymer container: filling 0.9× (brimful volume) of 0.1 mol/l HCl-solution (hydrochloric acid 30% Suprapur® (Merck)) diluted with demineralized water (ultrapure water of purity 1 analogue DIN ISO 3696 with ≤0.1 μS/cm at 25° C.) and isopropanol (1:1/vol %:vol %), i.e. the same volume as used in the alkaline treatment, in the pharmaceutical container;
closing the container with a closure seal;
inserting the container in an autoclave (e.g. Systec, model DX-150 (PM-CA-0001-01));
treating the container for 6 hours at 121° C. and 1 bar above ambient pressure;
opening the closure seal;
in case the container is a glass container: analyzing the content (mg/l) of one or more type(s) of ions and/or compounds, preferably [Na] ions, in the 0.1 mol/l HCl-solution by FAAS analysis, e.g. using Varian SpectrAA 280 FS (PE 3-004), to obtain the value [mg/l] for the leaching of one or more type(s) of ions and/or compounds, preferably [Na] ions;
in case the container is a polymer container: analyzing the content (mg/l) of one or more type(s) of ions and/or compounds, preferably the antioxidant and/or organic compounds, more preferably the antioxidant, in the 0.1 mol/l HCl-solution by LC-MS, e.g. using Waters I-Class UPLC system with Waters Xevo qTOF, to obtain the value [mg/l] for the leaching of one or more type(s) of ions and/or compounds, preferably the antioxidant and/or organic compounds, more preferably the antioxidant;
calculating the value (mg/cm²) by the following formula:

$$A * B / C ,$$

wherein A is the above value [mg/l] for the leaching of one or more type(s) of ions and/or compounds,
wherein B is 0.9× brimful volume, and
   wherein C is the wetted inner surface of the container (in cm²); to obtain the value [mg/cm²] for the leaching of one or more type(s) of ions and/or compounds, preferably Na ions or the antioxidant, i.e. the value i.

An alternative embodiment comprises a method (method Lᵢ), comprising the steps:
providing a container, preferably a coated container;
in case the container exhibits two openings, e.g. a syringe: covering the smaller opening with a closure, e.g. a tip cap;
filling 0.9× (brimful volume) of a 0.005 mol/l KOH-solution (KOH: Potassium hydroxide hydrate ≥99.995%, Suprapur® (Merck), demineralized water (ultrapure water of purity 1 analogue DIN ISO 3696 with ≤0.1 μS/cm at 25° C.)) in the pharmaceutical container;
closing the container with a closure seal, e.g. aluminum foil or a stopper;
inserting the container in an autoclave (e.g. Systec, model DX-150 (PM-CA-0001-01));
treating the container for 3 hours at 121° C. and 1 bar above ambient pressure, e.g. 2 bar;
opening the closure seal;
emptying the container;
filling and rinsing the container two times with deionized water;
filling 0.9× (brimful volume) of 0.1 mol/l HCl-solution (hydrochloric acid 30% Suprapur® (Merck)) diluted with demineralized water (ultrapure water of purity 1 analogue DIN ISO 3696 with ≤0.1 μS/cm at 25° C.)), i.e. the same volume as used in the alkaline treatment, in the pharmaceutical container;
closing the container with a closure seal;
inserting the container in an autoclave (e.g. Systec, model DX-150 (PM-CA-0001-01));
treating the container for 6 hours at 121° C. and 1 bar above ambient pressure;
opening the closure seal;
emptying the container;
filling and rinsing the container two times with deionized water;
drying the container, preferably with air;
filling 0.9× (brimful volume) of n-hexane, i.e. the same volume as used in the alkaline treatment, in the pharmaceutical container;
shaking the pharmaceutical container in an orbital shaker (80 rpm) for 30 min at room temperature;
analyzing the content (mg/l) of one or more type(s) of ions and/or compounds, preferably the antioxidant and/or organic compounds, more preferably the antioxidant, in the n-hexane-solution by LC-MS, e.g. using Waters I-Class UPLC system with Waters Xevo qTOF, to obtain the value [mg/l] for the leaching of one or more type(s) of ions and/or compounds, preferably the antioxidant and/or organic compounds, more preferably the antioxidant;
calculating the value (mg/cm²) by the following formula:

$$A * B / C ,$$

wherein A is the above value [mg/l] for the leaching of one or more type(s) of ions and/or compounds,
wherein B is 0.9× brimful volume, and
wherein C is the wetted inner surface of the container (in cm²); to obtain the value [mg/cm²] for
the leaching of one or more type(s) of ions and/or compounds, preferably Na ions or the antioxidant, i.e. the value i.

The value o may be obtained by several methods and preferably the value o is determined as described herein. The value o is either the content (mg/cm²) of one or more type(s) of ions and/or compounds of the base material of the coated container and/or, preferably or, obtainable by the method Lₒ as described herein, more preferably the value o is obtainable by the method Lₒ as described herein.

If the same but uncoated container, which is used to obtain value i is available, then value o may be obtainable by method Lᵢ described above using the uncoated container to obtain the content (mg/cm²) of one or more type(s) of ions and/or compounds of the base material of the coated container. If the values i and o should be obtained by only one container, value i is obtainable be the method Lᵢ above and value o is obtainable by the method Lₒ below. Preferably, o is obtainable by the method Lₒ. The method Lₒ can be used for a container, which is coated or uncoated on the outside, however, preferably the container is uncoated on the outside. Preferably, in the methods Lᵢ and Lₒ, the biggest opening faces upwards.

### Method Lₒ

In one embodiment of this disclosure, the following method (method Lₒ) is provided, comprising the steps:
providing a container, preferably the same container as used for the method Lᵢ above;
in case the container exhibits two openings, e.g. a syringe: covering the smaller opening with a closure, e.g. a tip cap;
filling 0.9× (brimful volume) of a solution in the pharmaceutical container;
closing the container with a closure seal;
inserting the container in a beaker, e.g. a stainless steel or aluminum beaker;
filling the beaker with a 0.005 mol/l KOH-solution (KOH: Potassium hydroxide hydrate ≥99.995%, Suprapur® (Merck), demineralized water (ultrapure water of purity 1 analogue DIN ISO 3696 with ≤0.1 μS/cm at 25° C.)) till a level is reached which is the same as the level in the container;
closing the beaker with a closure;
inserting the beaker in an autoclave (e.g. Systec, model DX-150 (PM-CA-0001-01));
treating the beaker for 3 hours at 121° C. and 1 bar above ambient pressure, e.g. 2 bar;
opening the closure of the beaker;
emptying the beaker;
filling and rinsing the beaker two times with deionized water and washing the outside of the container by rinsing the outer surface with deionized water;
inserting the container again in the beaker, e.g. the stainless steel or aluminum beaker;
in case the container is a glass container: filling the beaker with a 0.1 mol/l HCl-solution (hydrochloric acid 30% Suprapur® (Merck)) diluted with demineralized water (ultrapure water of purity 1 analogue DIN ISO 3696 with ≤0.1 μS/cm at 25° C.)))) till a level is reached which is the same as the level in the container, i.e. the same volume as used in the alkaline treatment, or
in case the container is a polymer container: filling the beaker with a 0.1 mol/l HCl-solution (hydrochloric acid 30% Suprapur® (Merck)) diluted with demineralized water (ultrapure water of purity 1 analogue DIN ISO 3696 with ≤0.1 μS/cm at 25° C.) and isopropanol (1:1/vol %:vol %), till a level is reached which is the same as the level in the container, i.e. the same volume as used in the alkaline treatment;
closing the beaker with a closure;
inserting the beaker in an autoclave (e.g. Systec, model DX-150 (PM-CA-0001-01));
treating the beaker for 6 hours at 121° C. and 1 bar above ambient pressure;
opening the beaker;
in case the container is a glass container: analyzing the content (mg/l) of one or more type(s) of ions and/or compounds, preferably [Na] ions, in the 0.1 mol/l HCl-solution by FAAS analysis, e.g. using Varian SpectrAA 280 FS (PE 3-004), to obtain the value [mg/l] for the leaching of one or more type(s) of ions and/or compounds, preferably [Na] ions;
in case the container is a polymer container: analyzing the content (mg/l) of one or more type(s) of ions and/or compounds, preferably the antioxidant and/or organic compounds, more preferably the antioxidant, in the 0.1 mol/l HCl-solution by LC-MS, e.g. using Waters I-Class UPLC system with Waters Xevo qTOF, to obtain the value [mg/l] for the leaching of one or more type(s) of ions and/or compounds, preferably the antioxidant and/or organic compounds, more preferably the antioxidant;
calculating the value (mg/cm²) by the following formula:

$$A * B / C ,$$

wherein A is the above value [mg/l] for the leaching of one or more type(s) of ions and/or compounds,
wherein B is the volume of the solution in the beaker, and
wherein C is the wetted outer surface of the container (in cm²); to obtain the value [mg/cm²] for the leaching of one or more type(s) of ions and/or compounds, i.e. the value o.

### Methods and Definitions

### ToF-SIMS (Time-of-Flight Secondary-Ion-Mass-Spectrometry)

In the following the measuring method and the data evaluation of the specific TOF-SIMS measurement is explained in detail. The values for the ions, e.g. the ions [C₃H₅⁺], [C₄H₃⁻], [SiC₂H₃⁺] and [SiCH₃O⁻], can be obtained according to the following description.

### Measuring Method

For the measurement a TOF SIMS (TOF.SIMS 5 from lontof) can be used. If not stated otherwise, the TOF-SIMS are measured according to ASTM E 1829 und ASTM E 2695.

The measurement has been started after establishing a vacuum of 1.0 mbar or less.

The following parameter settings were used for the TOF-SIMS:

### For the Analysis:

primary ion: Bi³⁺;
energy: 30 keV;
measuring area: 100×100 μm²;
pattern: 128×128 random; and
bismuth analysis current: 0.3 pA.

### For the Sputter Gun (Argon Cluster Source):

sputter ion: Ar-cluster;
energy: 10 keV;
sputter area: 300×300 μm²; and
sputter current of Ar-cluster source: 8.5 nA.

### Further Experimental Details:

cycle time: 200 μs
analyzer extractor: 2462 V (positive ions) and 2480 V (negative ions);
analyzer detector: 9000 V;
charge compensation: flood gun;
primary ion flight time correction: on; and
gas flooding: 9×10⁻⁷ mbar.

A sample of a coated polymer container, for example one half of an on the inside coated container, which was cut lengthwise in two pieces, is positioned in such a way that the centerline of the sputter gun and the centerline of the liquid metal ion gun of TOF-SIMS hit the coated area of the sample so that the sputter area covers the entire measuring area, preferably that the centerline of the sputter gun and the centerline of the liquid metal ion gun of TOF-SIMS hit the same point of the coated area of the sample. The TOF-SIMS measures either positive ions or negative ions. To obtain both kinds of ions, two measurements can be conducted using for each measurement a new area of the same sample or a new sample, e.g. the first and the second half of a coated container, which is cut lengthwise in two pieces.

### Determination of the Layers

The layer structure of the coated element includes, in this order, a smoothing layer (SL), a barrier layer (BL), a protection layer (PL), and an optional gliding layer (GL), wherein the coated element has a polymer substrate. Each of the said layers is chemically distinct and is characterised by the detection of specific ToF-SIMS species, i.e. SiC⁻, C₄⁻C₄H₃⁻, SiCH₃O⁻, and SiO₃⁻ in negative mode, and C₃H₅⁺, SiO⁺. SiC₂H⁺, SiC₂H₃⁺, Si₂O⁺, SiC₃H₉+, and CH₃⁺ in positive mode. The exact qualitative and quantitative characteristics depend on both the PICVD parameters employed during each of the four deposition steps (1 to 4) in addition to the chosen precursor, and the sputter and analysis parameters of the ToF-SIMS measurement. FIG. 1B and FIG. 1C, respectively, show the characteristic trend of the point-to-point normalisation intensity of selected ToF-SIMS species in positive and negative mode.

### Boarder of Layers

The order of layers built on top of the polymer substrate, in this order, are a smoothing layer (SL), a barrier layer (BL), a protection layer (PL), and an optional gliding layer (GL). Based on Example 1, and using the above sputter and analysis ToF-SIMS settings, the boarder(s) between the smoothing layer (SL), the barrier layer (BL), the protection layer (PL), and the optional gliding layer (GL), may be found or established on one or more of the following criteria:

$${[ {SiCH}_{3} {O}^{-} ]}_{GL} / {[ {SiO}_{3}^{-} ]}_{GL} > 1. ;$$

$${[ {SiCH}_{3} {O}^{-} ]}_{BL} / {[ {SiO}_{3}^{-} ]}_{BL} < 1. ;$$

$${[ {SiC}_{3} {H}_{9}^{+} ]}_{GL} / {[ {SiO}^{+} ]}_{GL} > 1.1 ;$$

$${[ {SiC}_{3} {H}_{9}^{+} ]}_{PL} / {[ {SiO}^{+} ]}_{PL} < 1. ;$$

$${[ {SiO}^{+} ]}_{BL} / {[ {SiC}_{2} {H}^{+} ]}_{BL} > 1.1 ;$$

$${[ {SiO}^{+} ]}_{GL} / {[ {SiC}_{2} {H}^{+} ]}_{GL} < 1.1 ;$$

$${[ {SiO}^{+} ]}_{PL} / {[ {SiC}_{2} {H}^{+} ]}_{PL} < 1.1 ; and$$

$${[ {SiO}^{+} ]}_{SL} / {[ {SiC}_{2} {H}^{+} ]}_{SL} < 1.1 .$$

Once the polymer substrate has been reached, i.e. after sputtering away all of the smoothing layer (SL), the barrier layer (BL), the protection layer (PL), and the optional gliding layer (GL), C₃H₅⁺ and C₄H₃⁻ as well as C₄⁻ become the dominant ToF-SIMS species in positive and negative mode, whose point-to-point normalisation (intensities) are >>1.0 as compared to all Si-containing ToF-SIMS species.

### Thickness of the Coating and its Layers

The thickness of the coating, as well as the individual layers, i.e. the smoothing layer, the barrier layer, the protection layer, and the gliding layer, is estimated by TEM (transmission electron microscopy). A Scanning Transmission Electron Microscope (SETM; Hitachi HF5000) is used at an accelerating voltage of 200 kV, at a capture resolution of 1024×1024 and an acquisition time of 20 seconds. Further instrument settings are: column mode operation (HR), DF-mode (ZCM), BF-mode (WAM), and STEM condenser aperture #2.

Sample preparation is conducted by first sputtering metallic titanium layer of 50 nm, to establish conductivity and surface protection, using a cold magnetron. Subsequent steps are performed using an Auriga 40 SEM/FIB cross beam: SEM-induced deposition of carbon was done using an organic precursor (for the protection from Ga ions), FIB-induced deposition of Pt using an organic precursor (for the protection of the surface during lamella preparation), at 30 kV Ga FIB to cut the lamella, sample transfer to a copper grid, thinning with 30 kV Ga ions, and final thinning with 2 kV Ga ions. This typically results in a final lamella width of 20 μm, and a final thickness of the titanium layer ca. 50 nm.

### Roughness of the Uncoated Surface of the Element

The surface roughness depth SRz has been determined based on and according to ISO 25178-2:2012 using a white light interferometer (WLI) on an uncoated inner surface of the element as well as an uncoated outer surface of the element. A WLI from Zygo (type NexView) was used, and a field-of-view of 400 μm×400 μm was measured and corrected for the curvature of the element before any of the subsequent calculations. To obtain SRz parameters, a surface area was analyzed by fitting a minimum enclosing rectangle and applying a 5×5 sampling grid, for a total of 25 sampling areas. All sampling areas together make up the evaluation area. Whereas Rz refers to the roughness obtained from an individual line scan (across 5 points), SRz reflects the average radial peak-to-valley areal roughness on a surface, i.e. the average of the largest half of many individual Rz results is determined by slicing the areal data array about its center through 360 degrees. The Rz results are sorted by magnitude and SRz is calculated by averaging the largest 50% of the Rz values. A line-generation algorithm is used to determine the actual pixel-to-pixel path of each slice, and there is no interpolation between pixels. SRz thus covers the entire array, and due to its radial generation it is lay independent.

Depending on the type of container, several measurements were performed and averaged over a well-defined number of containers and a well-defined number of measured positions.

For a syringe (COC, 1 ml long), 10 containers were measured at 6 different positions, giving a SRz of 132 nm±109 nm (mean±standard deviation). The 6 different positions were measured at a distance from the stamp of 10 mm, 25 mm, and 40 mm, and at opposite positions of 180° with respect to the central axis of the syringe.

For a vial (COP, 2 ml), 10 containers were measured at 3 different positions, giving a SRz of 147 nm±118 nm (mean±standard deviation). The 3 different positions were measured at a vial height of 10 mm and at angular positions of 120° with respect to the central axis of the vial.

### Gliding Force

Coefficients of static and gliding friction for a gliding layer were determined by pressing a V9361 FM457/0 FLNC2 057 stopper from Datwyler Pharma Packaging with an external diameter of 6.9±0.1 mm into a corresponding syringe at a speed of 100 mm/min. The forces required for this pressing action were recorded.

The coefficients of static and sliding friction are ascertained by moving a suitable stopper, immediately after insertion into the hollow body, at a constant speed of 100 mm/min through the hollow body and measuring the force required for this as a function of the insertion depth. Typical static/sliding friction diagrams have a linear rise in force at the start of movement of the stopper. As soon as the force of static friction has been overcome, the stopper begins to move and there is sliding of the stopper through the cylinder which, in the case of a good sliding layer, requires a relatively constant pushing force. Immediately prior to onset of the sliding movement of the stopper there is generally a maximum in the static/sliding friction diagram, which represents the coefficient of static friction. The relatively constant force during the sliding movement represents the coefficient of sliding friction. The static/sliding friction diagrams should typically be measured with a stopper that is also used as the stopper for the primary pharmaceuticals packaging and with which a corresponding leak tightness is achieved, without however requiring too great an effort.

### Breaking Force

The breaking force shall be understood as the static friction (force) measured at the surface of a coated element or an uncoated element.

The breaking force was measured in 1 ml long COC syringes with a siliconized stopper from Datwyler Pharma 257/2 ISAF2. The breaking force was measured as the maximal force along the initial path of 4 mm. After that initial path was passed, monitoring over the further path of 30 mm gave access to an average gliding force. Measurement was performed under dry conditions, after storing the stopper for 24 hours in the syringe at a temperature of 20° C. Further test parameters were: 100 N load cell, 100 mm/min test speed, plunger rod without thread.

### EXAMPLES

### Example 1 (Inventive Example)

A resin syringe (1 ml long COC) was provided and was coated subsequently in four steps without a vacuum break using an apparatus according to WO 2007/022976 A2 (FIG. 1). The plasma was driven by a microwave irradiation having a frequency of 2.45 GHz. The plasma reaction chamber was mainly the inside of the syringe and ambient conditions prevailed outside of the syringe. Before starting the film depositions the reaction chamber was evacuated until a value of 0.05 mbar was reached.

Four separate, subsequent coating steps were applied:

In a first step a gas flow of 20 sccm HMDSO was adjusted at a pressure of 0.66 mbar. Afterwards a plasma was excited with an input power of 1460 W in a pulsed mode with a pulse duration of 0.5 ms and pulse pause of 10 ms which equates to a duty cycle of 4.8%. The plasma treatment was performed for 5 seconds.

In a second step a gas mixture of 0.6 sccm HMDSO and 39.4 sccm Oxygen was adjusted at a pressure of 0.36 mbar. Afterwards a plasma was excited with an input power of 1180 W in a pulsed mode with a pulse duration of 2 ms and pulse pause of 5 ms which equates to a duty cycle of 28.6%. The plasma treatment was performed for 9 seconds.

In a third step a gas mixture of 14 sccm HMDSO and 6 sccm Oxygen was adjusted at a pressure of 0.66 mbar. Afterwards a plasma was excited with an input power of 1420 W in a pulsed mode with a pulse duration of 0.5 ms and pulse pause of 10 ms which equates to a duty cycle of 4.8%. The plasma treatment was performed for 1 seconds.

In a fourth step a gas flow of 10 sccm HMDSO was adjusted at a pressure of 0.84 mbar. Afterwards a plasma was excited with an input power of 980 W in a pulsed mode with a pulse duration of 0.04 ms and pulse pause of 1 ms. The plasma treatment was performed for 9 seconds. At the end the reaction chamber was vented with nitrogen until atmospheric pressure was reached.

### Example 2 (Inventive Example)

A resin syringe (1 ml long COC) was provided and was coated according to Example 1, except that the fourth coating step was omitted. A further coating was performed by employing a liquid coating according to Example 2 of EP 3636303 A1. The liquid coating was prepared by charging 80 g of a vinyl-functionalized polydimethylsiloxane in a reaction vessel and admixing with 640 g of hexamethyldisiloxane. Under constant stirring at 1000 rpm, 2 g of methylhydrosiloxane/dimethylsiloxane copolymer, 48 g of liquid polydimethylsiloxane, 1.1 g of 1,1,3,3-tetramethyl-1,3-divinyldisiloxane-complexed platinum and 0.1 g of butynol as inhibitor were added to this reaction mixture. The preparation can be used after a stirring time of 60 s.

The preparation was applied to the plastics hollow body on the inner side by a wiping process and cured by heating to 250° C. for 3.5 seconds. A 1-ml COC syringe of the “1-ml long” standard size with an internal diameter of 6.5 mm was used as substrate for depositing the gliding layer.

Coefficients of static and gliding friction for the cured gliding layer were subsequently determined. This involved pressing the V9361 FM457/0 FLNC2 057 stopper from Datwyler Pharma Packaging having an external diameter of 6.9±0.1 mm into the syringe at a speed of 100 mm/min. The forces required for this were recorded. The coefficients for both static and gliding friction were less than 10 N.

### Reference Example

A resin syringe (1 ml long COC) was provided and was coated according to Example 1, except that the fourth coating step was omitted.

What is claimed is:

1-20. (canceled)

21. A coated element having a surface, wherein at least a part of the surface of the element is coated by a coating, wherein the coating comprises, in this order:
a smoothing layer (SL);
a barrier layer (BL); and
a protection layer (PL),
wherein the smoothing layer satisfies the condition a1_{SL}≤[Si₂O⁺]_{SL}/[SiC₃H₉⁺]_{SL}≤a2_{SL}, wherein a1_{SL} is 1.0, wherein a2_{SL} is 1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, or
wherein the barrier layer satisfies the condition a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, or
wherein the protection layer satisfies the condition a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS.

22. The coated element as recited in claim 21 wherein the coated element has a gliding force of 0.1 to 10.0 N, or a breaking force of 1 N to 18 N.

23. A coated element having a surface, wherein at least a part of the surface of the element is coated by a coating, wherein the coating comprises, in this order:
a smoothing layer (SL);
a barrier layer (BL); and
a protection layer (PL),
wherein the smoothing layer has a thickness of 100 nm to 2000 nm, or
wherein the barrier layer has a thickness of 5 nm to 500 nm, or
wherein the protection layer has a gliding force of 0.1 to 10.0 N, or a breaking force of 1 N to 18 N, or
wherein the coated element has a gliding force of 0.1 to 10.0 N, or a breaking force of 1 N to 18 N.

24. The coated element as recited in claim 21 further comprising a gliding layer (GL),
wherein the gliding layer is adjacent to the protection layer,
wherein the gliding layer satisfies the condition a1_{GL}≤[SiC₃H₉⁺]_{GL}/[Si₂O⁺]_{GL}≤a2_{GL}, wherein a1_{GL} is 1.1, wherein a2_{GL} is 1*10⁵, wherein [SiC₃H₉⁺]_{GL} are the counts of [SiC₃H₉⁺] ions in the gliding layer, measured by a TOF-SIMS; and wherein [Si₂O⁺]_{GL} are the counts of [Si₂O⁺] ions in the gliding layer, measured by a TOF-SIMS.

25. The coated element as recited in claim 21 wherein the coated element comprises glass or polymer.

26. The coated element as recited in claim 25 wherein the coated element is cyclic olefin copolymer or cyclic olefin polymer or polypropylene.

27. The coated element as recited in claim 21 wherein the gliding layer satisfies one or more of the following parameters:
a1_{GL}≤[SiC₃H₉⁺]_{GL}/[Si₂O⁺]_{GL}≤a2_{GL}, wherein a1_{GL} is 1.1, wherein a2_{GL} is 1*10⁵, wherein [SiC₃H₉⁺]_{GL} are the counts of [SiC₃H₉⁺] ions in the gliding layer, measured by a TOF-SIMS; and wherein [Si₂O⁺]_{GL} are the counts of [Si₂O⁺] ions in the gliding layer, measured by a TOF-SIMS, and
b1_{GL}≤[SiC₂H⁺]_{GL}/[SiO⁺]_{GL}≤b2_{GL}, wherein b1_{GL} is 1.1, wherein b2_{GL} is 1000, wherein [SiC₂H⁺]_{GL} are the counts of [SiC₂H⁺] ions in the gliding layer, measured by a TOF-SIMS; and wherein [SiO⁺]_{GL} are the counts of [SiO⁺] ions in the gliding layer, measured by a TOF-SIMS.

28. The coated element as recited in claim 21 wherein the protection layer satisfies one or more of the following parameters:
a1_{PL}≤[Si₂O⁺]_{PL}/[SiC₃H₉⁺]_{PL}≤a2_{PL}, wherein a1_{PL} is 1.0, wherein a2_{PL} is 1*10⁵, wherein [SiC₃H₉⁺]_{PL} are the counts of [SiC₃H₉⁺] ions in the protection layer, measured by a TOF-SIMS; and wherein [Si₂O⁺]_{PL} are the counts of [Si₂O⁺] ions in the protection layer, measured by a TOF-SIMS, and
b1_{PL}≤[SiC₂H⁺]_{PL}/[SiO⁺]_{PL}≤b2_{PL}, wherein b1_{PL} is 0.5, wherein b2_{PL} is 1*10⁵, wherein [SiC₂H⁺]_{PL} are the counts of [SiC₂H⁺] ions in the protection layer, measured by a TOF-SIMS; and wherein [SiO⁺]_{PL} are the counts of [SiO⁺] ions in the protection layer, measured by a TOF-SIMS.

29. The coated element as recited in claim 21 wherein the barrier layer satisfies one or more of the following parameters:
a1_{BL}≤[Si₂O⁺]_{BL}/[SiC₃H₉⁺]_{BL}≤a2_{BL}, wherein a1_{BL} is 1, wherein a2_{BL} is 1000, wherein [SiC₃H₉⁺]_{BL} are the counts of [SiC₃H₉⁺] ions in the barrier layer, measured by a TOF-SIMS, and wherein [Si₂O⁺]_{BL} are the counts of [Si₂O⁺] ions in the barrier layer, measured by a TOF-SIMS, and
b1_{BL}≤[SiO⁺]_{BL}/[SiC₂H⁺]_{BL}≤b2_{BL}, wherein b1_{BL} is 1, wherein b2_{BL} is 1*10⁵, wherein [SiC₂H⁺]_{BL} are the counts of [SiC₂H⁺] ions in the barrier layer, measured by a TOF-SIMS, wherein [SiO⁺]_{BL} are the counts of [SiO⁺] ions in the barrier layer, measured by a TOF-SIMS.

30. The coated element as recited in claim 21 wherein the smoothing layer satisfies one or more of the following parameters:
a1_{SL}≤[Si₂O⁺]_{SL}/[SiC₃H₉⁺]_{SL}≤a2_{SL}, wherein a1_{SL} is 1.0, wherein a2_{SL} is 1*1000, wherein [SiC₃H₉⁺]_{SL} are the counts of [SiC₃H₉⁺] ions in the smoothing layer, measured by a TOF-SIMS, wherein [Si₂O⁺]_{SL} are the counts of [Si₂O⁺] ions in the smoothing layer, measured by a TOF-SIMS, and
b1_{SL}≤[SiC₂H⁺]_{SL}/[SiO⁺]_{SL}≤b2_{SL}, wherein b1_{SL} is 1.0, wherein b2_{SL} is 1*10⁵, wherein [SiC₂H⁺]_{SL} are the counts of [SiC₂H⁺] ions in the smoothing layer, measured by a TOF-SIMS, wherein [SiO⁺]_{SL} are the counts of [SiO⁺] ions in the smoothing layer, measured by a TOF-SIMS.

31. The coated element as recited in claim 21 wherein the thickness of the smoothing layer is 1 nm to 20000 nm.

32. The coated element as recited in claim 21 wherein a time a sputter gun needs to reach the surface of the element is 0.5 minutes to 150 minutes or
wherein in the case positive ions are measured, the point, when the sputter gun reaches the surface of the element is the point, when the counts of [Si₂O⁺] ions are equal to the counts of [C₃H₅⁺] ions; or
wherein in the case negative ions are measured, the point, when the sputter gun reaches the surface of the element is the point, when the counts of [C₄H₃⁻] ions are equal to the counts of [SiO₃⁻] ions.

33. The coated element as recited in claim 21 wherein the coated element is a pharmaceutical container comprising an inner surface, wherein the inner surface is coated, and
wherein the smoothing layer is in direct contact with the coated element.

34. The coated element as recited in claim 24 wherein the gliding layer is the outermost layer.

35. The coated element as recited in claim 21 wherein one or more of the following conditions is/are fulfilled:
$${[ {SiO}_{3}^{-} ]}_{GL} < {[ {SiO}_{3}^{-} ]}_{BL} ;$$
$${[ {SiO}_{3}^{-} ]}_{PL} < {[ {SiO}_{3}^{-} ]}_{BL} ;$$
$${[ {SiO}_{3}^{-} ]}_{SL} < {[ {SiO}_{3}^{-} ]}_{BL} ; and / or$$
$${[ {SiO}_{3}^{-} ]}_{GL} < {[ {SiO}_{3}^{-} ]}_{PL} ,$$
wherein [SiO₃⁻]_{GL} are the counts of [SiO₃⁻] ions in the gliding layer, measured by a TOF-SIMS;
wherein [SiO₃⁻]_{PL} are the counts of [SiO₃⁻] ions in the protection layer, measured by a TOF-SIMS;
wherein [SiO₃⁻]_{BL} are the counts of [SiO₃⁻] ions in the barrier layer, measured by a TOF-SIMS;
wherein [SiO₃⁻]_{SL} are the counts of [SiO₃⁻] ions in the smoothing layer, measured by a TOF-SIMS.

36. A system or kit, comprising:
the coated element as recited in claim 21 wherein the coated element is a syringe or cartridge; and
a plunger.

37. A closed system comprising:
the coated element as recited in claim 21 wherein the coated element is a syringe or cartridge;
a plunger; and
a closure,
wherein the closed coated element passes the container closure integrity test according to DIN EN ISO 8871-5:2016; chapter 4.4 in combination with Annex D.

38. The closed system as recited in claim 37 further comprising a pharmaceutical composition.

39. The closed system as recited in claim 37 further comprising a composition containing biologics or mRNA.

40. A method for coating an element, comprising the steps of:
(1) providing an element comprising a surface;
(2) applying a smoothing layer;
(3) applying a barrier layer;
(4) applying a protection layer; and
(5) optionally applying a gliding layer,
wherein each of the steps (2) to (4) comprises surrounding at least part of the surface of the element with a precursor, and
wherein each of the steps (2) to (4) comprises irradiating the precursor to generate a plasma using a PICVD method,
wherein the step (3) comprises establishing a flow rate of O₂ of 10 sccm or more during the irradiation of the precursor.

41. The method as recited in claim 40 wherein the PICVD method is carried out in such a way that
(i) the irradiation is carried out by a microwave generator; or
(ii) wherein an input power IP is 100 W to 10000 W.

42. The method as recited in claim 40 wherein the PICVD method is carried out in such a way that
(i) the irradiation is carried out by a microwave generator, wherein the ray has a frequency of 2.45 GHz; or
(ii) wherein an input power IP is 900 W to 4000 W.

42. The method as recited in claim 40 wherein the precursor is HMDSO.

43. The method as recited in claim 40 wherein the application of the smoothing layer is performed at a ratio [ms/ms] of all pulse durations PD_{SL} [ms] to all pulse pauses PP_{SL} [ms] of 0.01 to 0.5, the application of the barrier layer is performed at a ratio [ms/ms] of all pulse durations PD_{BL} [ms] to all pulse pauses PP_{BL} [ms] is 0.01 to 1, or the application of the protection layer is performed at a ratio [ms/ms] of all pulse durations PD_{PL} [ms] to all pulse pauses PP_{PL} [ms] is 0.001 to 1.
